(19) United States
(12) Patent Application Publication
(10) Pub. No.: US 2014/0335193 A1
(43) Pub. Date: Nov. 13, 2014

(54) CONTROLLED-RELEASE INJECTABLE MICROPARTICLE

(71) Applicants: Ignacio Rintoul, Santa Fe (AR); Juan Manuel Badano, Santa Fe (AR); Ricardo Grau, Santa Fe (AR)

(72) Inventors: Ignacio Rintoul, Santa Fe (AR); Juan Manuel Badano, Santa Fe (AR); Ricardo Grau, Santa Fe (AR)

(73) Assignees: CONICET - CONS. NAC. DE INVESTIGACIONES CIENTIFICAS Y TECNICAS, Buenos Aires (AR); IPCVA- INSTITUTO DE PROMOCION DE LA CARNE VACUNA ARGENTINA, Buenos Aires (AR); UNL UNIVERSIDAD NACIONAL DEL LITORAL, Sante Fe (AR); Gerardo Rintoui, Alicante (ES)

(21) Appl. No.: 14/117,508

(22) PCT Filed: May 11, 2012
(86) PCT No.: PCT/ES12/70336

§ 371 Date: May 1, 2014

(30) Foreign Application Priority Data
May 13, 2011 (AR) 20110101665

(51) Int. Cl. A61K 9/14; A61K 9/00; A61K 31/57

(52) U.S. Cl. CPC A61K 9/146; A61K 31/57; A61K 9/0019
USPC 424/501; 514/177; 427/2.21

(57) ABSTRACT

The invention relates to a controlled-release injectable microparticle comprising a polyvinyl alcohol polymer and one or more hormones, in particular progesterone. Said microparticle induces estrus in female mammals after a single application. The invention also relates to a method for obtaining the microparticle.

### TECHNICAL FIELD OF THE INVENTION

This invention belongs to the field of microparticles for the controlled release of veterinary actives. Polyvinyl alcohol microparticles of adequate size for injectable applications are of special interest. Likewise, this invention provides a method for obtaining these microparticles by dripping an aqueous solution into another aqueous solution.

Furthermore, the present invention is related to a formulation useful for the control of oestrus in livestock animals by means of a single application of the microparticle of the present invention to induce ovulation. The present invention refers also to the method of control of oestrus, according to which female cattle can be inseminated between days 7 and 15 subsequent to the application of the microparticles of this invention.

### STATE OF THE ART

90% of production animal reproduction is performed without human intervention. [1] This is due, in great measure, to the impossibility of keeping a strict control over the animal to detect the initiation of the oestrous cycle as a step prior to the artificial insemination process. As an example, it is stated that in beef cattle the fertile period of reproductive females barely ranges from 24 to 48 hours, with the inconvenience that within the same herd there are animals that enter into heat with differences of several weeks or months. This implies that the personnel in charge of doing the insemination should be in attendance practically every day, sample each animal one by one to detect the ones in heat and eventually inseminate them. In addition, the difficulties of accessing herds whether because of the long distances between their location and town centres, bad road conditions, and the logistics themselves necessary for the activities that are carried out in the open field, all increase the associated costs, turning animal reproduction assisted by artificial insemination into a non-profitable method. As a result, considerable delays are verified in increasing the genetic load of herds, with the ensuing loss of productivity. Parturition rates have time differences of weeks or months, which undoubtedly make the assistance, control, and development of newborns highly inefficient as regards vaccination and feeding programs, planting of forages, operating grazing fields, and making effective use of personnel involved. [2]

The following are the main advantages that oestrous cycle synchronisation would provide in production animals: [3]

1) Concentrating animals in oestrus within a short period of time;

2) Rationalising artificial insemination, mainly in beef cattle;

3) Concentrating and reducing the parturition period and rationalising vaccination programs;

4) Handling available feed based on the time of year and the categories of animals;

5) Facilitating the design of a zootechnical assessment test to enable the purchase of individuals at reduced intervals between births;

6) Registering heifers, facilitating handling and commercialisation practices;

7) Increasing genetic improvement rates in breeds and herds.

Several technologies have been developed with the aim of simultaneously synchronising the oestrous cycle in several reproductive females. They all imply the administration of drugs and hormones with various functions in the stimulation of the reproductive cycle of the animals. Release matrices have been employed for the sustained administration of drugs and hormones, i.e., to maintain a certain concentration of the drug or drugs in the animal over a certain period of time. These matrices are drug reservoirs generally made from a polymeric material, and they have the property of steadily releasing the drug over a certain period of time. The polymeric matrices can adopt different forms and may be placed in different sites on the animal's body to activate its function.

The most extended system is the intravaginal route. Intravaginal devices have been developed in different forms. Below is a brief description of such devices and their manufacturing concepts.

The PRID-type intravaginal device (Progesterone-Releasing Intravaginal Device) is constituted by 1.55 to 2.25 grams of micronised progesterone uniformly suspended in a silicon matrix that, in turn, has been cured on a stainless steel coil. Its manufacture consists in the injection moulding of a progesterone mix suspended in liquid silicone on a stainless steel plate of roughly 3.5×28.5×0.1 cm³. Then the silicone is left to cure inside the mould until it becomes semisolid elastic, upon which it is removed from the mould and a coil of approximately 4 cm in diameter and some 12 cm in length is formed. Further information about the production and functioning of PRID devices may be obtained from the following citations:
[4,5,6,7,8,9,10,11,12,13,14,15,16,17,18,19,20,21,22,23,24,25,26,27,28,29,30,31,32,33,34,35,36, 37,38,39,40,41,42,43,44,45,46,47,48,49,50,51,52,53,54,55,56,57,58,59,60,61,62,63,64,65,66, 67,68,69,70,71].

The CIDR-B-type intravaginal device consists in a T- or Y-shaped nylon core on which a layer of around 0.9 to 5.0 mm silicone impregnated in 1.9 grams of micronised progesterone is deposited by injection moulding. Further information about the production and functioning of CIDR-B devices may be obtained from the following citations:
[72,73,74,75,76,77,78,79,80,81,82,83,84,85,86,87,88,89,90,91,92,93,94,95,96,97,98,99,100, 101,102,103,104,105,106,107,108,109,110,111,112,113,114,115,116,117,118,119,120,121,122, 123,124,125,126,127,128,129,130,131]

A multitude of sponge-type intravaginal devices have been developed and studied academically. Nevertheless, very few have reached the serial production stage and been turned into commercial products. Developed sponges feature different lengths, diameters, densities, porosity, and consistency. In general, sponge bases are obtained through processes similar to the ones used to obtain expanded polyurethane. In fact, expanded polyurethane is the most used material to obtain this type of intravaginal device. These processes consist in the dissolution of the polymer in a liquid solvent with a low boiling point. Then the solvent is rapidly volatised, forming during this process a sponge from the polymer initially dissolved. Countless variants of this process have been developed. Subsequently, the intravaginal sponge is obtained by pulverising a solution containing a certain amount of hormone in a volatile solvent, for example, alcohol, chloroform, acetone or its mixes, on the surface of the sponge obtained previously. Through this method, sponges impregnated with one or several of the following hormones have been obtained: progesterone, fluorgesterone acetate, norethandrolone, melengestrol acetate, chlormadione acetate, megestrol acetate, methylacetoxyprogesterone, and aestradiol, all of which have proven participation in regulating the reproductive cycle. Once the sponges are impregnated in hormones, the solvent is left to evaporate and the hormones remain finely deposited on the large surface of the sponge. An alternative method is the immersion of the sponge base in a hormone solution bath. After immersion, the sponge is removed from the bath and the solvent is left to evaporate, whereby a fine dispersion of hormones is obtained on the large surface of the sponge. The addition of antibiotics is common in these intravaginal devices because an increase in the specific surface of the device increases the likelihood of microbial colonisation. Further information about the production and functioning of sponge-type intravaginal devices may be obtained from the following citations:
[132,133,134,135,136,137,138,139,140,141,142,143].

The INVAS-type intravaginal device (Intravaginal Application System) consists in a flexible T-shaped polypropylene structure roughly 145 mm in length and coated with a silicone skin where 1.42 grams of progesterone are found homogeneously dispersed. The shape of the INVAS is similar to the CIDR-B device. However, the technology associated with each one of them is what sets them apart. In particular, the curing method of the silicone matrix is the main difference. In the INVAS case, incorporating progesterone in the silicone matrix is carried out by means of a laminating and rolling process similar to the one used when incorporating sulphur and black smoke to rubber in tire manufacturing. In this case, the material is treated in paste form. Once the hormone is intimately dispersed within the silicone paste, the latter is laminated to a fine sheet or film of some 2 to 10 mm in thickness. Then it is die cut in a T-shape and a film sandwich with a polypropylene film flexible silicone structure is formed inside a mould. The mould is closed and heated around 70° to 120° C. to cure the silicone and seal the sandwich structure. This method enables the use of low-fusion point plastics, which in the case of the CIDR-B device and the progesterone does not change its crystalline structure during the process. On the other hand, the process time frame necessary to obtain the INVAS device is sensibly longer than to obtain the CIDR-B device. Further information about the production and functioning of INVAS-type intravaginal devices may be obtained from the following citations: [144, 145].

The ring-type intravaginal device was developed unsuccessfully. This type of intravaginal device consists in a steel or thermoformed plastic ring coated in a hormone-impregnated silicone skin. In effect, the greatest inconvenience of this type of intravaginal device is its low degree of retention in the vaginal cavity, which in no case was higher than three days. Further information about production and functioning of ring-type intravaginal devices may be obtained from the following citation: [146].

The Rajamehendran-type intravaginal device is obtained from two silicone tubes of about 20 cm in length and with inner and outer diameters of 0.79 cm and 1.27 cm, respectively. One end of both tubes is sealed with a silicone adhesive. Subsequently, progesterone dissolved in diethyl ether is introduced into the tubes through the open ends. Once the diethyl ether has evaporated, these ends are sealed with silicone adhesive. Then an aestradiol paste mixed with silicone adhesive is spread in a layer on the ends of the silicone tubes. Finally, both tubes are cross-tied together at mid-length. A series of threads or strings may be fastened to the tubes to facilitate their removal from the vaginal cavity. Further information about the production and functioning of Rajamehendran-type intravaginal devices may be obtained from the following citations: [147,148,149].

IBD-type intravaginal devices (Intelligent Breeding Devices) enable the release of various hormones (estadiol, prostaglandin, and progesterone) at perfectly determined rates and time intervals. These devices consist in a head attached to a container. The head has flexible tubular ramifications that facilitate the introduction of the device, its retention in the vaginal cavity, and the release of the hormones. The container is a rigid plastic tube of around 12 cm in length, 4 cm in diameter and hermetically closed. There is an integrated circuit, micropumps, hormone reservoirs, and a set of batteries in the interior. This circuit is programmed in such a way that it indicates the speed and duration at which the different hormone loads should be introduced into the animal. Further information about the production and functioning of IBD-type intravaginal devices may be found from the following citation: [150].

Every intravaginal device seeks to achieve a steady release of one or more hormones with the aim of encouraging the induction of oestrus in animals. These devices need to be introduced into the animal and reach the uterus to activate its function. Their large size and geometric complexity make the production, storage, and transportation of intravaginal devices difficult, and their use implies the following steps:

1) Immobilising the animal to have access to its hind quarters. This task is not always easily attainable in the precariousness of the open field.

2) Washing the vaginal area of the animal to minimise the risk of infections. It is indispensable to use gloves, cleaning agents, disinfectants and, above all, an abundant availability of clean water, which many times is difficult to obtain in the field.

3) Changing gloves between one animal and another to avoid the propagation of vaginal infections and, more important, to avoid any contact between the surface of the device and the operator's skin, which could lead to transferring part of the device's hormone load to the operator's body. Then the device should be very carefully inserted into the vaginal cavity of the animal. This task should be performed by duly specialised personnel, which implies extra costs in education and training. On the other hand, the need for serial production in these devices makes it practically impossible to manufacture custom-made devices, so there is always the risk of an under- or overdose of hormones, or of devices failing to enter or entering loosely and falling out of the vagina. It should be mentioned that the vaginas of animals have great variations in size, physical consistency, and specific features unique to each one of them, depending on their size, breed, age, previous parturitions, etc. The amount of hormone required varies from animal to animal.

4) The device should be removed after a period that may vary between 7 to 15 days. To achieve this, the animal should be immobilised and washed again. The device should be removed by specialised personnel in line with appropriate safety measures, as it is common for there to be a residual amount of hormone in the device ranging between 40 and 60%.

5) Finally, the used device should be buried or burned as final disposal measures to avoid any future accidental contact.

Ordinarily, intravaginal devices are used in conjunction with complementary hormone intramuscular applications. The following is a description of a protocol for use of an intravaginal device produced and commercialised by Pfizer:

Day 0—1^{st} action: immobilising the animal; 2^{nd} action: sanitising the vaginal area; 3^{rd} action: placing the intravaginal device; 4^{th} action: intramuscular injection of 2 mg of aestradiol.

Day 8—5^{th} action: immobilising the animal; 6^{th} action: sanitising the vaginal area; 7^{th} action: removing the intravaginal device; 8^{th} action: injecting a dose of prostaglandin.

Day 9—9^{th} action: injecting aestradiol; 1 mg in cows and 0.75 in heifers.

Day 10—10^{th} action: inseminating upon detected oestrus or fixed-time artificial insemination.

Furthermore, the burning or burying of the device is required for its final disposal. Intravaginal devices release around 0.6 mg of progesterone on a daily basis. It should be noted that between the start of the treatment and the day of insemination, every animal has been handled 10 times, making treatment-related labour costs high.

Given this situation, and for reasons of complexity and inefficiency inherent in intravaginal devices, the technology has been oriented toward the development of other release systems using subcutaneous routes. This release route can be classified into two general branches: implantable and injectable systems. Implantable systems generally require minor surgery to gain access to subcutaneous tissue, where the release system is to be implanted. Injectable systems gain access to these tissues by means of veterinary needles.

Release systems implantable under the skin were one of the first subcutaneous release systems developed. These systems consist in tubes, spheres, slabs or discs made of silicone, hydron or other biocompatible, but not necessarily biodegradable, polymer, which have been loaded somehow with progesterone or another hormone. These systems are implanted under the animal's skin by means of a small surgical procedure to allow for the direct release of the hormone or hormones into the animal's organism. In these cases, the quality and purity of the hormone used, as well as the size and the form of the implant, are of crucial importance. The effectiveness of these release systems is in general quite good. Nevertheless, the problem of dosage remains. Implants are manufactured with a single hormone load. Yet the amount of hormones necessary to induce oestrus varies according to differences in breeds, sizes, and the age of the animals. In some cases, additional surgery is required to extract exhausted systems. The large size of the implants, the need to immobilise the animal in order to carry out the surgery, the need for aseptic conditions specific to surgery, and the eventual double sequence of tasks linked to the removal of the exhausted implant leads one to seriously doubt the feasibility of implementing this type of release system. Further information about the production and functioning of implantable subcutaneous release systems may be obtained from the following citations: [151,152,153,154,155].

Implantable subcutaneous release systems under the dewlap have certain advantages in relation to subcutaneous implants. These advantages are mostly related to the ease with which surgery may be performed in this area of the animal where the hide is thin. These systems are conceived in the form of silicone bars containing one or several hormones. The surface of these bars may vary between 15 and 4 cm² with hormone content close to 500 milligrams. However, a great amount of residual hormone remains inside the device without being released to the implant site. In this sense, it has been proven that implants with a higher ratio of surface-volume are more effective in the release of hormones, but difficulties in implantation are increased. Aside from facilitating surgery, this technique has practically the same inconveniences than subcutaneous implants, i.e., the need to immobilise the animal, the need to aseptically accommodate the area where surgery is to be performed, the implant surgery, dosage problems, and the eventual removal of the exhausted implant. Further information about the production and the functioning of implantable release systems under the dewlap may be obtained from the following citations: [156,157,158,159].

Implantable subcutaneous release systems in the ear were conceived as a way to facilitate operations to implant and remove implants. Various types of implants in the ear and their applicable implanter devices have been developed at a commercial scale. These implants are polymer tubes, such as Hydron, polyurethane or silicone, of around 3 mm in diameter by 20 mm in length, and contain between 5 and 15 mg of hormones homogeneously distributed in the polymer matrix. Implants are stored in protector seals. Once the seal is opened, the implant is extracted and placed in the implanter device, and with the help of this device, it is introduced in the animal's ear with relative ease, thereby enormously simplifying the task of surgery. Many of these implants are accompanied by intramuscular aestradiol injections. The removal of the implant is performed by means of a small incision and is relatively easy provided that it has been placed in the middle of the ear. However, as in the cases previously cited, the amount of residual hormone is between 50 and 65%; hence, the dosage problem remains and it has been proven that the rate at which the hormone is released into the animal's organism is not steady during treatment. In an attempt to keep a constant rate of release, MDD technology (Microsealed Drug Delivery) was developed. This type of release system is similar to the previously mentioned one with the only difference that the hormone is contained in micro-reservoirs of polyethylenglycol dispersed in the polymer matrix. Further information about production and functioning of implantable subcutaneous release systems may be obtained from these citations:
[160,161,162,163,164,165,166,167,168,169,170,171,172,173,174,175,176, 177].

Injectable systems are an alternative to eliminating surgical procedures used to implant and remove implantable release systems and the dosage problem. An application by injection does not require rigorous aseptic conditioning of the area where the injection is to be applied; the application of the injection is much simpler and quicker than by implant and it is possible to dose each animal in particular by controlling the volume injected. In this type of system, it is impossible to remove the release system once exhausted, so it is necessary to design it based on matrices that are absolutely biocompatible and eventually biodegradable. Injectable release systems are classified in three categories: transdepot, preformed systems, and in-situ formed system.

Injectable transdepot release systems consist in liquid hormone solutions with variable viscosity where the hormone or hormones are dispersed or dissolved. The solution is always in a liquid state when being injected. Once injected, the solution becomes semisolid as a result of a physical-chemical change induced by the environment of the injection site. This technology was developed with applications in human medicine. Then it was transferred to veterinary medicine. However, this constitutes a major inconvenience for its dissemination. Human medicine may admit much higher costs than veterinary medicine. Therefore, in most cases, the simple transfer of the application from humans to animals results in a more or less technical success, but highly unfeasible from an economic standpoint. The so-called thermoplastic pastes are injected as molten liquid at a higher temperature than the one in the injection site. Once injected, the liquid cools off at the animal's temperature, forming a semisolid and entrapping the hormone load in its interior. The hormone load will be released to the medium according to the structure and characteristics of the depot. The most used materials for the manufacture of these systems are low molecular weight biocompatible and biodegradable polymers and copolymers derived from lactic acid, glycolic acid, caprolactone, trimethylene carbonate, dioxanone, and other esters. Nevertheless, it is important to stress that many times, it is possible to fuse these polymers at a temperature higher than 60° C., whereby its injection may be very painful to the animal and produces necrosis and eschars in its immediacies. In addition, this implies the use of a thermostabilisable syringe. Another disadvantage is its relatively low drug release rate. Further information about the production and functioning of injectable thermoplastic paste release systems may be obtained from the following citations:
[178,179,180,181,182,183,184,185,186,187,188,189].

In-situ reticulation systems are another type of injectable transdepot release systems. The concept is based on the preparation of a liquid solution of monomers, polymers, reticulation agents, and the drug load that can be dissolved or dispersed in said solution. This solution is injected into the animal. Once injected, the reticulant agent is activated by any physical-chemical stimulus, polymerising and joining polymeric chains, thus forming a semisolid with the drug load entrapped in its interior. The drugs will be released to the medium according to the system's structure and characteristics. The systems are manufactured from the materials mentioned above. It is usual to add acrylic monomers and polymers, and to use peroxides or N,N-dimethyl-p-toluidine as initiators of the reticulation. These initiators should be added to the solution seconds before their injection, to avoid having the reticulation process take place inside the syringe. There are various forms of stimulating reticulation. Polymerisation by free radicals and curing are the most common. Nevertheless, these types of systems are limited due to the toxicity of the monomers and the reticulation agents, to the inhibition capacity in the reaction of physiological conditions and body fluids, and to the rise in temperature due to strongly exothermic reactions of polymerisation and curing that seriously affect the surrounding tissues. Further information about the production and functioning of injectable reticulation type release systems may be obtained from the following citations:
[190,191,192,193,194,195,196,197, 198,199,200,201,202,203].

The most recent injectable transdepot-type release systems are the so-called ion-induced gelling systems. These systems are built from biopolymers that have the capacity to gel when in contact with multivalent cationic ions. Alginates and albumins, in combination with calcium, are the most disseminated. The injection process is similar to the previous one. Further information about the production and functioning of injectable ion-induced gelling release systems may be obtained from the following citations: [204,205,206,207,208,209,210,211,212,213].

Finally, injectable release systems formed from polymer precipitation should be mentioned. In these cases, a biocompatible and biodegradable polymer mix, generally based on lactic acid, glycolic acid, caprolactone and others, a biocompatible and bioassimilable solvent, such as N-methyl-2-pyrrolydone, propylenglycol, dimethyl sulfoxide, tetrahydrofuran, 2-pyrrolydone, among others, and the hormone load. This mix should be homogeneous and stable while it is stored. Once injected, polymer precipitation is induced in the injection site, forming a solid or semisolid structure with the drug entrapped in its interior. The drug will be released to the medium according to the characteristics of the structure formed. In general, the polymer is precipitated by solvent removal, changes of temperature, or changes in pH. The addition of different types and amounts of tenso-active agents, such as Tween80 and Span80, allows for the control of the polymer precipitation in the form of massive elements, sponges, particulates and micro particulates. Limitations for this type of procedures are the high level of drug released right before and in the first phases of polymer precipitation. This causes serious irritations in the surrounding tissue and can even be toxic for the receptor organism. The effects in the organism caused by the majority of solvents and eventually the tenso-active agents mentioned, especially dimethyl sulfoxide and N-methyl-2-pyrrolydone, are also highly controversial. They are toxic and can damage muscular activity if administered orally, intraperitoneally and intravenously. There is no information on their effects at the subcutaneous level. Others are potentially hemolytic, with necrotic capability. Further information about the production and functioning of injectable precipitation-based polymer release systems may be obtained from the following citations: [214,215,216,217,218,219,220,221,222,223,224,225,226,227,228,229,230,231,232,233,234, 235,236,237,238,239,240,241,242,243,244,245,246,247,248,249].

The situation described above was a driving force for the development of injectable subcutaneous release systems consisting in preformed microparticles loaded with hormones. A microparticle is a general term that encompasses microspheres and microcapsules. The term micro is used to describe systems capable of flowing and being injected by means of veterinary needles of roughly 1,000 microns or less. The technique consists in the preparation of generally spheric microparticles. The hormone load is entrapped or encapsulated during the process of obtaining the microparticles themselves. Then these microparticles are dispersed in an excipient liquid. Finally, a certain volume of this fluid is taken with a certain volume of microparticles and injected into the animal.

The following is a very brief explanation of the fundamentals of each technique:

Encapsulation by Emulsion: The technique consists in dispersing two immiscible phases with the help of surfactant agents, and where the content only is soluble in the discontinued phase. Subsequently, the continuous phase can be extracted by means of dry spray techniques, and the content remains encapsulated in the dispersed phase. [250,251]

Encapsulation by Internal Gelling: The technique consists in dissolving the content and usually a polysaccharide in water. Then this solution is dispersed in an immiscible oily phase that contains a polysaccharide gelling precursor dissolved in it. With the addition of a destabiliser, generally Ca++, gelling in the dispersed phase is generated, and the content is entrapped (encapsulated) in this gelled matrix. [252,253,254]

Encapsulation by Phase Separation: The technique consists in forming a stable suspension of the content by means of an aqueous polymer. Then polymer precipitation is induced, and the content is entrapped inside the precipitated particles. [255,256]

Encapsulation by Interfacial Polymerisation: The technique consists in dissolving or dispersing the content in a monomer solution. Then this solution is dispersed in an oily phase, in which an immiscible polymerisation initiator is added in the monomeric phase. Polymerisation occurs in the interface, and the content is encapsulated in the interior of the monomeric phase. [257,258]

Encapsulation by In-situ Polymerisation: The technique consists in dispersing the content in an immiscible phase with a dissolved monomer. Then only the continuous phase is polymerised, and the content is encapsulated in a polymeric matrix. [²⁵⁴]

Encapsulation by Atomisation: The technique consists in dispersing a polyelectrolyte solution, usually alginate sodium, in a solution of gelling salts, usually calcium chloride. Then the precipitates are dispersed in a polyelectrolyte solution with the opposite charge, usually polylysine. [259]

Encapsulation by Desolvation: The technique consists in dissolving the content and the material forming the membrane in a small amount of solvent. Then this solution is extruded or dispersed in a medium with excess of non-solvent liquids. This non-solvent desolvates the solution, and the content is entrapped in a matrix formed by the material forming the membrane. [²⁵⁵]

Encapsulation by Centrifugal Extrusion: The technique consists in pumping the content and the material forming the membrane through a double-headed rotor, the content through the inner head and the material through the outer head. The centrifugal force breaks down the flow into droplets of content coated by the material forming the membrane. These droplets turn into capsules upon entering into contact with the gelling agent solution of the material forming the membrane. [260]

Encapsulation by an Atomising Rotating Disc: The technique consists in dispersing the content in a liquid film on a rotating disc. When the fluids reach the edge of the disc, they are expelled in the form of droplets of content coated with the material forming the membrane. These droplets turn into capsules upon entering into contact with the gelling agent solution. [261,262]

Encapsulation by Jet Cut: The technique consists in dissolving the content and the material forming the membrane. This solution is made to go through a sieve, forming a continuous jet.

Then this continuous jet is sectioned by means of a rotor with several wires. The sections then fall into a gelling agent solution, forming capsules. [263]

Encapsulation by Electrostatic Dripping: The technique consists in dissolving the content and the material forming the membrane, and then this solution is made to go through a needle and finally falls into a gelling agent solution. A difference in potential between the needle and the gelling agent solution helps to expel the droplets, releasing them from the needle tip. [264,265]

Encapsulation by Vibratory Extrusion. The technique consists in dissolving the content and the material forming the membrane to later make this solution go through a vibrator head and finally fall into a gelling agent solution. The vibration helps to expel the droplets, releasing them from the head. [266]

Polyvinyl alcohol-based microparticles have been prepared using microencapsulation techniques by emulsion [267,268,269,270,271,272,273,274,275], interfacial polymerisation [276,277], inner gelling [278,279], and spray-drying [280]. No information has been found as to obtaining polyvinyl alcohol-based microparticles by dripping. Nor has any information been found as to the use of polyvinyl alcohol as a matrix for hormone release. Hormone microencapsulation using polymers derived from lactic acid, glycolic acid, or the like, as a matrix for release has been reported [281,282,283,284,285,286,287,288,289].

### BRIEF DESCRIPTION OF THE INVENTION

The object of this invention is an injectable controlled-release microparticle that comprises a polyvinylalcohol polymer and one or several hormones. Said microparticle is characterised because the polyvinylalcohol polymer presents a hydrolysis figure higher than 85%, preferably higher than 90%, and more preferably higher than 95%. In addition, the polyvinylalcohol polymer of the mentioned microparticle has a viscosity according to Din 53015 protocol between 5 and 110 mPa·s, preferably between 20 and 110 mPa·s, more preferably between 20 and 70 mPa·s. In a preferred embodiment of this invention, the value of polymer viscosity is between 30 and 50 mPa·s.

The hormone of the microparticle of the invention is selected from a group that comprises progesterone and its variants, aestradiol and its variants, prostaglandins and their variants, all the variants of prostanoic acid, steriods with progestagen activity, such as MGA, melengestrol acetate, CAP (6-chloro-6-dehydro-17α-acetoxy-pregn-4-ene-3.20-dione), MAP (6α-methyl-17α-acetoxy-pregn-4-ene-3.20-dione); blocks of progestagen, such as norgestomet, aestradiol valerate, aestradiol benzoate, 17 α aestradiol, gonadotropins such as GnRH, LH, CG, PMSG, FSH; and mixes of said hormones. In a preferred embodiment of the present invention, the hormone is progesterone. The progesterone load in the microparticle of the invention has a mass value ranging between 5 and 70%. In another of the preferred embodiments, the progesterone load of the microparticle of the invention has a value in between 50 and 70%. In another of the preferred embodiments, the progesterone load of the microparticle is at least 5%. The progesterone-release microparticle of this invention, in addition, consists in a diameter of said microparticle ranging between 0.2 and 5 mm, preferably a diameter between 1.5 and 2.5 mm, and dispersion in the diameters between 0.01 and 0.1 mm. In one of the preferred forms of embodiment of this invention, said microparticle comprises in a diameter ranging between 1 and 2 mm when the hormone load is between 5 and 40% in weight, and sphericity between 1 and 1.5; in another of the preferred embodiments of this invention, said progesterone-releasing microparticle comprises in a diameter between 2 and 2.5 mm when the hormone load is between 40 and 50% in weight, dispersion in the diameters between 0.01 and 0.1 mm, and sphericity between 1 and 1.5.

Another object of this invention is the process for obtaining said microparticles, which comprises by the following steps:

a- Preparing an aqueous solution A, of polyvinylalcohol and hormone to be encapsulated;

b- Preparing an aqueous solution B of sodium hydroxide, with the optional adding of additives;

c- Dispersing solution A within solution B;

d- Stabilising the microparticles formed in step c, leaving them in suspension, for a period between 2 and 90 minutes, and at a temperature between 20 and 90° C.;

e- Recovering the microparticles, separating them from solution B;

f- Drying the microparticles in a controlled atmosphere at a temperature between 25 and 120° C. under conditions of a fluidised bed to achieve the removal of excess solvation water;

g- Conditioning the microparticles.

Step a- The preparation of an aqueous solution A comprises mixing in the same container polyvinylalcohol (PVA) between 5 and 50% in weight, glycerol (GL) between 0.05 and 1% in weight, boric acid (BH) between 0.05 and 5% in weight and progesterone between 5 and 70% in weight. The mix is softly stirred in a thermostated bath at a temperature between 10 and 90° C. for 5 to 240 minutes until total dissolution of the PVA, BH and GL. Step b- for the preparation of an aqueous saline solution (solution B), using sodium hydroxide or another equivalent base ranging between 0.05 and 1% in weight. Step c- for the dispersion of solution A in solution B consists in dripping solution A by gravity feed into solution B at a volumetric ratio ranging between 5 to 50 parts of solution B for each part of solution A. Step c-, in addition, is performed by dripping using a drip head, and solution A droplets are released from the drip head by gravity feed. In another of the forms of embodiment of this invention, in Step c-, solution A droplets are released by vibratory action. In another embodiment, said vibration is induced mechanically, and in another of the forms of embodiment, said vibration is induced by sound. In another embodiment, said vibration is induced mechanically, and in another of the forms of embodiment, said vibration is induced by means of electric or piezoelectric coils activated by alternating currents. In another of the forms of embodying this invention, Step c- for the dispersal of solution A in solution B is performed by dripping using a drip head, and the solution A droplets are released with electrostatic assistance. In another of the forms of embodying this invention, Step c- for the dispersal of solution A in solution B is performed by dripping using a drip head, where solution A droplets are released with the assistance of blowing with a gaseous current, preferably air.

The process for obtaining the microparticles in this invention also comprises a step subsequent to Step c-, for microparticle stabilisation, that is performed within aqueous solution B during the stabilisation period, with the adding of stabilisers or stabilising agents. In a form of embodiment of this invention, Step d- is performed within aqueous solution B for 2 to 90 minutes at a temperature between 20 and 90° C.

In said process for obtaining the microparticle of this invention, the step for recovering the microparticles is performed by means of a method selected from the group comprised by flotation, sedimentation, centrifugation or filtration, and is followed by a step for washing with a stabilising solution.

In one of the preferred forms of embodiment of the process in this invention, the step for the microparticle recovery is subsequent to the step for microparticle stabilisation and it consists in at least one of the following steps:

filtration, sedimentation, centrifugation, or flotation;

modification of the stabilising solution;

reduction of volume of the stabilising solution.

In this invention, the preferred form for microparticle recovery is by filtration.

In the process of this invention, furthermore, the step for microparticle conditioning comprises at least 3 optional steps:

I. removal of the solvation water remaining in the microparticles by hot air drying at a temperature between 25 and 120° C. under conditions of a fluidised bed;

II. dispersion of the microparticles in 2-pyrrolydone;

III. spraying with an aqueous glycerol solution between 10 and 60% in weight on the surface of the microparticles and subsequent exposure of the microparticles to a temperature ranging between 100 and 120° C. and a pressure between 20 and 100 bar.

The administration of the described microparticles to a female mammal to induce oestrus is another object of this invention. In addition, said microparticles induce oestrus in said female mammal after a single application.

### DETAILED DESCRIPTION OF THE INVENTION

The method for obtaining microparticles for the release of drugs consists in 6 basic or fundamental stages:

1) Preparing an aqueous solution containing polyvinyl alcohol, reticulation or gelling additives, and the drug or drugs to be released. This solution is named solution A.

2) Preparing an aqueous saline solution with the eventual adding of other additives. This solution is named solution B.

3) Dispersing solution A into solution B.

4) Stabilising the microparticles.

5) Recovering the microparticles.

6) Conditioning the microparticles.

The microparticles obtained may be injected into animals for veterinary purposes, or eventually in humans for medical purposes.

1) Preparing an aqueous solution containing polyvinyl alcohol, reticulation or gelling additives, and the drug or drugs to be released. This solution is named solution A. Solution A may be prepared in different ways. The PVA of this solution must have a viscosity evaluated according to norm DIN 53015, between 5 and 110 mPa·s, preferably between 20 and 70 mPa·s, more preferably between 30 and 50 mPa·s. Three forms of preparing solution A are mentioned as examples.

1a) Appropriate quantities of water, PVA, additives and drugs are added for obtaining solution A. The mix is stirred until a major part of the PVA and the additives is dissolved. A coadjuvant or surfactant may be necessary. The drug should remain practically insolubilised in the medium. Help from heat, or heat and pressure, may eventually be used to speed up the dissolution process. The maximum temperature used should always be, as a minimum, less than the temperature at which the least stable compound in the mix starts to decompose or denaturalise.

1b) A first component of the mix is dissolved in the total volume of water. Once it has dissolved, a second component is added to be dissolved, and likewise with the rest of the components, which are all added gradually and dissolved one by one until solution A is obtained.

1c) PVA and the additives are both dissolved separately in water. The drug is dispersed in water. A coadjuvant or surfactant may be necessary. Then these three solutions are mixed to obtain the final solution A. The help of heat, or of heat and pressure, may eventually be used to speed up the various dissolution and/or dispersion processes, provided that the properties of the mix components are not altered.

2) Preparing an aqueous saline solution with the eventual adding of other additives. This solution is named solution B. Solution B may be prepared in different ways. Three forms of preparing solution B are mentioned as examples.

2a) Appropriate quantities of water, salts, and additives are added to obtain solution B. The mix is stirred until a major part of the salts and additives are dissolved. A coadjuvant or surfactant may be necessary. The help of heat, or heat and pressure, may eventually be used to speed up the dissolution process. The maximum temperature used should always be less than the temperature at which the least stable compound in the mix starts to decompose or denaturalise.

2b) A first component of the mix is dissolved in the total volume of water. Once it has dissolved, the second component is added to be dissolved, and likewise with the rest of the components, which are all added gradually and dissolved one by one until solution B is obtained.

2c) Each one of the components of the mix are dissolved separately in water. A coadjuvant or surfactant may be necessary. Then these solutions are mixed to obtain solution B. The help of heat, or of heat and pressure, may eventually be used to speed up the various dissolution and/or dispersion processes, provided that the properties of the mix components are not altered.

3) Dispersion of solution A in solution B. Each solution A droplet dispersed in solution B forms a semisolid polyvinyl alcohol-based sphere, with the drug remaining entrapped in its interior. The dispersion of solution A in solution B is performed by dripping. Solution A is dripped into solution B. The dripping process may be performed in multiple ways. These ways basically differ in how the droplets are released from the tip of the drip head or heads. The drip head consists in one or multiple needles, a cluster of tubes, or even holes. Three forms of dripping are mentioned as examples.

3a) Traditional Dripping. This dripping is performed by gravity feed. Droplets are released from the head by gravity feed.

3b) Dripping Assisted by Vibration. In this dripping, the head is vibrated to facilitate the release of droplets. This vibration can be induced mechanically, by sound, or by means of electric or piezoelectric pumps activated with alternating currents.

3c) Electrostatic Dripping. In this dripping, an electrostatic force is added to gravity feed. To do so, a vertically oriented electrostatic field is generated. The droplets are released from the head by the joint action of gravity feed and the electrostatic force.

3d) Dripping Assisted by Blowing. In this case, the droplets are released from the head by using a gaseous current, generally air.

4) Microparticle Stabilisation. A microparticle stabilisation process originating in solution A and immersed in solution B after the dispersion process is commonly necessary. In some cases, microparticle stabilisation is fast enough to consider that it occurs during the dispersion process itself and will not require an additional process. The stabilisation process may be performed in various ways. Three forms of stabilisation are mentioned as examples.

4a) Microparticles formed from solution A and immersed in solution B are stabilised for a certain time in solution B itself.

4b) Microparticles formed from solution A and immersed in solution B are stabilised for a certain time in a modified solution B by adding stabilisers or stabilisation accelerating agents.

4c) Microparticles formed from solution A and immersed in solution B are recovered by sedimentation, flotation, centrifugation, or filtration. Once recovered, they are immersed or washed in another solution with stabilising capacity. Multiple washes, immersions, or combinations of both can be employed.

5) Microparticle Recovery. Once stabilised mechanically and chemically, the microparticles can be easily manipulated. Depending on the case, the microparticles can be recovered to go on to the following process, or otherwise, for their immediate use or storage. The recovery process can be performed in various ways. Three forms of recovery are mentioned as examples.

5a) Recovery by Filtration, Sedimentation, Centrifugation, or Flotation. In this type of recovery, the physical separation of the particles from the stabilising solution is sought by applying any of the methods mentioned above. The resulting microparticles can either go on to the following process, be injected or used immediately, or else be packaged or stored for their subsequent use.

5b) Recovery by Volume Reduction. In this case, a part of the stabilising solution can be extracted to concentrate the microparticles. Then the remaining solution can be modified to condition the mix of microparticles concentrated in this solution, and go on to the following process, be injected or used immediately, or else be packaged or stored for their subsequent use.

5c) Recovery by Modifying the Stabilising Solution. In this case, some physical change is induced in the microparticles by means of a chemical change in the stabilising solution. These changes include dehydration, densification, or solvent exchange in the microparticles. The microparticles in these conditions can go on to the following process, be injected or used immediately, or else be packaged or stored for its subsequent use.

6) Microparticle Conditioning. Microparticle conditioning is a general term that indicates the preparation of microparticles for their immediate application or storage. In this stage, the final characteristics of the microparticles are adjusted. The conditioning can consist in various processes or their combinations. Three types of conditionings are indicated below as examples.

6a) Bulk Conditioning. This conditioning refers to the interior of the microparticles. The interior of the microparticles can be conditioned by adjusting the residual water, inducing a chemical reaction, extracting a certain component present in the microparticles but undesirable for its application or storage, or adding a certain component desirable for its application or storage.

6b) Superficial Conditioning. This conditioning refers to the surface of the microparticles. The surface of the microparticles can be conditioned by inducing some chemical reaction, forming or eliminating membranes, extracting a certain component present in the surface of the microparticles but undesirable for its application or storage, or adding a certain component to the surface of the microparticles that may be desirable for its application or storage.

6c) Excipient Conditioning. An excipient is understood as a substance, commonly inactive, that is mixed with the microparticles to give it consistency, stability, fluidity, or another characteristic to the mix, or else, to facilitate some aspect or aspects of its use. The conditioning of the excipient can consist in adding excipient to a group of microparticles, or in modifying a mix of microparticles immersed in a stabilising or recovery solution.

### DETAILED DESCRIPTION OF AN EXAMPLE OF THE PROCESS TO OBTAIN MICROPARTICLES

Polyvinyl alcohol-based microparticles charged with progesterone with applications in the control of oestrus and ovulation of production animals

1) Preparing solution A. The strategy described in section 1a) above is used. An aqueous solution is prepared: PVA with an hydrolysis degree of 95%, 10% in weight, that could be between 5 and 50% in weight, plus its viscosity, evaluated according to norm DIN 53015, presenting a value of 45 mPa·s, that could be between 5 and 110 mPa·s; glycerol (GL) at 0.5%, that could be between 0.05 and 1% in weight; boric acid (BH) at 1%, that could be between 0.05 and 5% in weight; and progesterone at 5% that could be aggregated at between 5 and 70% in weight. All the components are weighed and mixed in the same container. The mix is stirred softly in a thermostated bath at a temperature of 30° C., although this phase could also be performed at a temperature between 10 and 90° C. during 25 minutes, being extendable up to 5 to 240 minutes until total dissolution of the PVA, BH and GL. The progesterone is dispersed in the mix given that it is practically insoluble in aqueous solvents.

2) Preparing solution B. The strategy described in section 2a above is used An aqueous solution of sodium hydroxide is prepared at 0.8%, ranging between 0.05 and 1% in weight being also possible. This dissolution is fast, and it does not need heat or other additives.

3) Dispersing solution A in solution B. The strategy described in section 3a above is used. Solution A is made to drip by gravity feed into solution B at a volumetric ratio of 10 parts of solution B for each art of solution A, that could be extended up to ranges from 5 to 50 parts of solution B for each part of solution A. Each solution A droplet that is submerged in solution B is transformed almost instantaneously into a semisolid microparticle. Dripping continues until solution A runs out.

4) Microparticle Stabilisation. The strategy described in section 4a above is used. Once dripping of solution A into solution B is concluded, the suspension of A microparticles in solution B is left under moderate stirring for a period of time of 20 minutes, being possible to extend it up to 2 to 90 minutes at a temperature of 25° C., that could be of between 20 to 90° C. Microparticles of 1.5 mm of diameter are thus obtained, that could be of between 0.2 to 5 mm of diameter and a dispersion in diameters of 0.04 mm, that could be of between 00.01 to 0.1 mm. Besides, this microparticles present a spherecity of 1.1, that could be the same of between 1 and 1.5.

5) Microparticle Recovery. The strategy described in section 5a above is used. The microparticles are recovered by filtration. The remaining solution B can be discarded or reconditioned to be reused in step 2.

6) Microparticle Conditioning. A combination of strategies 6a, 6b and 6c described above is used. Bulk conditioning consists in removing the remaining solvation water from the microparticles. The water is removed by hot air drying at a temperature at 50° C., that could also be of between 25 and 120° C. under conditions of a fluidised bed. Superficial conditioning consists in the formation of a membrane by superficial reticulation of the PVA chains. An aqueous glycerol solution is prepared at 20% in weight, that could also be of between 10 and 60% in weight. The solution is distributed on the surface of the microparticles by spraying. Then the system is subjected to a temperature of 100° C., that could also be ranging between 100 and 120° C. and a pressure of 50 bar, that could also be of between 20 and 100 bar. The system is cooled off and can be fractioned, packaged and stored. Conditioning the excipient consists in dispersing the microparticles in 2-pyrrolydone to provide fluidity to the system and to enable its passage through veterinary needles.

### DETAILED DESCRIPTION OF THE FORM OF APPLICATION

The microparticles of the present invention can release drugs with various applications. The microparticles are fractioned and stored in vials. From the vials, the microparticles are taken by means of a veterinary syringe to be then injected in animals. The type, quantity, and injection site will depend on each specific application. Eventually, they can be introduced into the digestive tract of the animals by means of hoses or applicators specifically designed for that purpose.

### DETAILED DESCRIPTION OF THE ADVANTAGES FOR THE CASE OF POLYVINYL ALCOHOL-BASED MICROPARTICLES CHARGED WITH PROGESTERONE WITH APPLICATIONS IN THE CONTROL OF OESTRUS AND OVULATION IN PRODUCTION ANIMALS IN COMPARISON TO OTHER TECHNOLOGICAL ALTERNATIVES

Advantages of a hormone release system by preformed microparticles of the present invention in comparison to intravaginal devices: Its small and uniform size facilitates its storage and transportation. There are no risks implicit in fortuitous contacts between the microparticles and the operator's skin. Microparticles are stored in vials, the doses are picked up by syringes, and are injected in the animals. At no time is there any risk of contact. As it is injectable, there is no need for immobilising the animal, or rigorous sanitising of the animal as required for the introduction of intravaginal devices. As rigorous hygiene is unnecessary, it is also unnecessary to have ample availability of clean water. By controlling the volume of microparticles injected, an optimum dose of the hormone load can be attained for each animal in particular. With the use of intravaginal devices, on the contrary, only one hormone load is available and there are always risks of an under- or overdose. Moreover, once the microparticles are injected, they remain in the animal without any risk of their falling out as in the case of intravaginal devices. The application of injectables can be performed by non-specialised personnel in comparison to the high cost of training required for the introduction of intravaginal devices. By using microparticles, 100% of the hormone load is used in comparison to intravaginal devices, where as little as 30% is used. It is not necessary to extract the exhausted microparticles, in comparison with the multiple tasks involved in immobilising, sanitising, extracting, and burning or burying the intravaginal devices.

Advantages of the hormone release system by preformed microparticles of the present invention in comparison to implantable systems under the skin or dewlap: The advantages of injectable preformed microparticles in comparison to implants are the ease of dosing the hormone load of the microparticles in comparison to the fixed hormone load of implants, the high content of residual hormone remaining in the implants, and no need for implant surgery and the eventual implant removal surgery with its implications as to the need of immobilising the animal, the decrease in infection risks, hygiene requirements and the training of the personnel involved.

Advantages of the hormone release system by preformed microparticles of the present invention in comparison to subcutaneous release systems implantable in the ear: The advantages of injectable preformed microparticles in comparison to implants in the ear are their ease of dosing in relation to the fixed dosing of implants and no need for implant removal surgery.

Advantages of the hormone release system by preformed microparticles of the present invention in comparison to transdepot-type injectable release systems: The advantages of the injectable preformed microparticles compared to transdepot devices are non-functionality with the temperature of the microparticles compared to thermoplastic pastes, a perfectly defined and reproducible release surface of the microparticles compared to irregular and unrepeatable forms and surfaces commonly observed in transdepot devices, the non-presence of toxic elements in the microparticles compared to the presence of surfactants, solvents, and other additives used in the transdepots, and the unpredictability in the hardening reactions of thermoplastic pastes, in-situ reticulation, gelling, and polymer precipitation with its implications as to the rate of hormone release.

Advantages of the hormone release system by preformed polyvinyl alcohol-based microparticles of the present invention compared to preformed microparticles based on polymers derived from lactic acid, glycolic acid, caprolactone and others: the main advantage is cost, because the technology already described in this document implies a diminish in costs of polymeric prime materials in the process of manufacture, of at least 50% in comparison with the use of other polymers.

### DETAILED DESCRIPTION OF THE NOVEL ASPECTS OF THE INVENTION

There is no prior information about obtaining microparticles by sequencing the processes described above and independently from its material base, type and quantity of drugs in its interior and field of application.

There is no prior information for obtaining polyvinyl alcohol-based microparticles for veterinary applications.

### REFERENCES

1 Censo Nac. Agropecuario-2007. INTA. www.inta.gov.ar.

2 Plan sanitario productivo. CAPROVE. www.caprove.gov.ar

3 Becaluba F. 2006. METODOS DE SINCRONIZACION DE CELOS EN BOVINOS. www.produccion-animal.com.ar

4 J. F. Roche. Control of oestrus in cattle. World Rev. Anim. Prod. 15 (1979) 49-76.

5 R. D. Smith, R. J. Pomerantz, W. E. Beale, J. P. McCann, T. E. Pilbeam, W. Hansel. Insemination of Holstein heifers at a preset time after etrus cycle synchronization using progesterone and prostaglandin. J. Anim. Sci. 58 (1984) 792-800.

6 J. F. Roche. Control of time of ovulation in heifers treated with progesterone and gonadotrophin releasing hormone. J. Reprod. Fertil. 43 (1975) 471-477.

7 V. W. Winkler, S. Borodkin, S. K. Webel, J. T. Mannebach. In vitro and in vivo considerations of a novel matrix controlled bovine progesterone-releasing intravaginal device. J. Pharm. Sci. 66 (1977) 816-818.

8 J. F. Roche. Retention rate in cows and heifers of intravaginal silastic coils impregnated with progesterone. J. Reprod. Fertil. 46 (1976) 253-255.

9 J. F. Roche. Fertility in cows after treatment with a prostaglandin analogue with or without progesterone. J. Reprod. Fertil. 46 (1976) 341-345.

10 J. F. Roche, J. J. Ireland. Effect of exogenous progesterone on time of occurrence of the LH surge in heifers. J. Anim. Sci. 52 (1981) 580-586.

11 J. F. Roche. Synchronisation of oestrus in cows using intravaginal silastic coils containing progesterone. Ann. Biol. Anim. Biochem. Biophys. 15 (1975) 301-302.

12 J. F. Roche. Control of oestrus in cattle using progesterone coils. Anim. Reprod. Sci. 1 (1978) 145-154.

13 R. E. Mauer, S. K. Webel, M. D. Brown. Ovulation control in cattle with progesterone releasing intravaginal device (PRID) and gonadotropin releasing hormone (GnRH). Ann. Biol. Anim. Biochem. Biophys. 15 (1975) 291-296.

14 J. F. Roche, D. J. Prendiville. Synchronisation of oestrus and pregnancy diagnosis in heifers bred in autumn and winter. Vet. Rec. 102 (1978) 12-14.

15 J. F. Roche. Comparison of pregnancy rate in heifers and suckler cows after progesterone or prostaglandin treatments. Vet. Rec. 99 (1976) 184-186.

16 J. F. Roche, D. J. Prenderville, W. D. Davis. Calving rate following fixed time insemination after a 12-day progesterone treatment in dairy cows, beef cows and heifers. Vet. Rec. 101 (1977) 417-419.

17 A. Shaham, Y. Folman, M. Rosenberg. Progesterone concentration in the reproductive tissues of dairy cows treated with a progesterone releasing intravaginal device (PRID). Proc. 12th Int. Cong. Anim. Reprod., 1992, pp. 1184-1186.

18 R. K. Munro. Concentrations of plasma progesterone in cows after treatment with 3 types of progesterone pessaries. Aust. Vet. J. 64 (1987) 385-386.

19 R. K. Munro. The effects of duration and concentration of plasma progesterone on the fertility of post-partum cows treated with pregnant mare serum gonadotrophin and intravagainal progesterone. Aust. Vet. J. 66 (1989) 43-45.

20 J. F. Roche. Calving rate of cows following insemination after a 12-day treatment with silastic coils impregnated with progesterone. J. Anim. Sci. 43 (1976) 164-169.

21 J. F. Roche, J. P. Gosling. Control of estrus and progesterone levels in heifers given intravaginal progesterone coils and injections of progesterone and estrogen. J. Anim. Sci. 44 (1977) 1026-1029.

22 N. A. Robinson, K. E. Leslie, J. S. Walton. Effect of treatment with progesterone on pregnancy rate and plasma concentrations of progesterone in Holstein cows. J. Dairy Sci. 72 (1989) 202-207.

23 Y. Folman, M. Kaim, Z. Herz, M. Rosenberg. Reproductive management of dairy cattle based on synchronization of estrus cycles. J. Dairy Sci. 67 (1984) 153-160.

24 Y. Folman, S. R. McPhee, I. A. Cumming, I. F. Davis, W. A. Chamley, Conception rates in cows after various synchronisation techniques using progesterone releasing intravaginal devices. Aust. Vet. J. 60 (1983) 44-47.

25 M. E. Wehrman, M. S. Roberson, A. S. Cupp, F. N. Kojima, T. T. Stumpf, L. A. Werth, M. W. Wolfe, R. J. Kittok, J. E. Kinder. Increasing exogenous progesterone during synchronization of estrus decreases endogenous 17b-es-tradiol and increases conception in cows. Biol. Reprod. 49 (1993) 214-220.

26 M. S. Roberson, M. W. Wolfe, T. T. Stumpf, R. J. Kittok, J. E. Kinder. Luteinizing hormone secretion and corpus luteum function in cows receiving two levels of progesterone. Biol. Reprod. 41 (1989) 997-1003.

27 A. Cupp, M. Garcia-Winder, A. Zamudio, V. Mariscal, M. E. Wehrman, N. Kojima, K. Peters, E. Bergfeld, P. Hernandez, T. Sanchez, R. Kittock, J. Kinder. Two concentrations of progesterone (P4) in circulation have a different effect on pattern of ovarian follicular development in the cow. Biol. Reprod. 45 (1992) 106.

28 S. R. McPhee, M. W. Doyle, I. F. Davis, W. A. Chamley. Multiple use of progesterone releasing intravaginal devices for synchronisation of oestrus and ovulation in cattle. Aust. Vet. J. 60 (1983) 40-43.

29 E. E. Custer, W. E. Beal, S. J. Wilson, A. W. Meadows, J. G. Berardinelli, R. Adair. Effect of melengestrol acetate (MGA) or progesterone-releasing intravaginal device (PRID) on follicular development concentrations of estradiol-17b and progesterone and luteinizing hormone release during an artificially lengthened bovine estrus cycle. J. Anim. Sci. 72 (1994) 1282-1289.

30 E. G. Bergfield, F. N. Kojima, M. E. Wehrman, A. S. Cupp, K. E. Peters, V. Mariscal, T. Shanchez, R. J. Kittok, M. Garcia-Winder, J. E. Kinder. Frequency of luteinizing hormone pulses and circulating 17b-oestradiol concentration in cows is related to concentration of progesterone in circulation when the progesterone comes from either an endogenous or exogenous source. Anim. Reprod. Sci. 37 (1995) 257-265.

31 D. C. Bulman, G. E. Lamming. Milk progesterone levels in relation to conception in repeat breeding and factors influencing acyclicity in dairy cows. J. Reprod. Fertil. 54 (1978) 447-458.

32 R. K. Munro, N. W. Moore. Effects of progesterone, oestradiolbenzoate and cloprostenol on luteal function in the heifer. J. Reprod. Fertil. 73 (1985) 353-359.

33 R. K. Munro, N. W. Moore. The use of progesterone administered intravaginally and pregnant mares serum gonadotrophin given by injection in controlled breeding programs in beef and dairy cattle. Aust. Vet. J. 62 (1985) 228-234.

34 R. K. Munro. Factors affecting oestrus response and calving rates following 7-day intravaginal progesterone treatment of cattle. Aust. Vet. J. 64 (1987) 192-194.

35 I. A. Cumming, S. R. McPhee, W. A. Chamley, Y. Folman, I. F. Davis. The time of oestrus and ovulation following various synchronisation techniques using progesterone impregnated intravaginal devices. Aust. Vet. J. 59 (1983) 14-18.

36 Y. Fukui, K. Mutoh, M. Tsubaki, I. Odagiri, Y. Masuto, H. Ono, H. Yagura. The use of a progesterone releasing intravaginal device (PRID) on synchronization of estrus in Japanese Black cattle. Jpn. J. Anim. Reprod. 30 (1984) 117-126.

37 S. Tjondronegoro, P. Williamson, G. J. Sawyer, S. Atkinson. Effects of progesterone intravaginal devices on synchronization of estrus in postpartum dairy cows. J. Dairy Sci. 70 (1987) 2162-2167.

38 Y. Fukui, M. Kobayashi, M. Tsubaki, N. Kikuchi, H. Ono. Regulating estrus and therapy of repeat-breeder and anestrus Holstein heifers using progesterone releasing intravaginal devices (PRID). Jpn. J. Vet. Sci. 47 (1985) 943-950.

39 H. Uehlinger, H. Binder, B. Hauser, P. Rusch, K. Zerobin. Hormonanalytischer vergleich der vaginaleinlagen CIDR® and PRID bei ovariektomierten kuhen. Schweiz. Arch. Tierheilk. 137 (1995) 81-86.

40 J. S. Stevenson, M. O. Mee. Pregnancy rates of Holstein cows after postinsemination treatment with a progesterone releasing intravaginal device. J. Dairy Sci. 74 (1991) 3849-3856.

41 Y. Folman, S. R. McPhee, I. A. Cumming. The effect of Estrumate followed by progesterone coils on oestrus synchronization and conception of post-partum beef and dairy cows. Anim. Reprod. Sci. 4 (1981) 117-126.

42 P. J. Broadbent, L. D. Tregaskes, D. F. Dolman, M. F. Franklin, R. L. Jones. Synchronization of estrus in embryo transfer recipients after using a combination of PRID or CIDR-B plus PGF 2a. Theriogenology 39 (1993) 1055-1065.

43 A. R. Peters, D. S. Hewitt, G. E. Lamming. The effect of exogenous progesterone on plasma LH and milk progesterone concentrations in multiple-suckling post-partum cows. Anim. Reprod. Sci. 6 (1983) 103-110.

44 D. C. Bulman, P. E. McKibbin, W. T. Appleyard, G. E. Lamming. Effect of a progesterone releasing intravaginal device on the milk progesterone levels, vaginal flora, milk yield and fertility of cyclic and non-cyclic dairy cows. J. Reprod. Fertil. 53 (1978) 289-296.

45 R. Rajamahendran, L. Forgrave, P. C. Lague, R. D. Baker. Control of estrus in heifers. Can. J. Anim. Sci. 55 (1975) 787.

46 D. H. Holness, D. H. Hale. The response of lactating Africander cows to treatment with a progesterone releasing intravaginal device or injection of synthetic GnRH. Anim. Reprod. Sci. 3 (1980) 181-188.

47 A. R. Ellicott, C. E. Thompson, J. R. Hill. Pregnancy rates in cows and heifers inseminated at predetermined times using progesterone-releasing intravaginal devices. Theriogenology 8 (1977) 315-321.

48 L. R. Sprott, J. N. Wiltbank, W. N. Songster, S. Webel. Estrus and ovulation in beef cows following use of progesterone releasing devices progesterone and estradiol valerate. Theriogenology 21 (1984) 349-356.

49 S. B. Drew, C. M. Gould, D. C. Bulman. The effect of treatment with a progesterone releasing intravaginal device on the fertility of spring calving Friesian dairy cows. Vet. Rec. 103 (1978) 259-262.

50 P. J. H. Ball, G. E. Lamming. Diagnosis of ovarian acyclicity in lactating dairy cows and evaluation of treatment with gonadotrophin-releasing hormone or a progesterone releasing intravaginal device. Br. Vet. J. 139 (1983) 522-527.

51 M. W. Smith, J. S. Stevenson. Fate of the dominant follicle, embryonal survival and pregnancy rates in dairy cattle treated with prostaglandin F 2a and progestins in the absence or presence of a functional corpus luteum. J. Anim. Sci. 73 (1995) 3743-3751.

52 W. E. Beal. A note on synchronization of oestrus in post partum cows with prostaglandin F 2a and a progesterone releasing device. Anim. Prod. 37 (1983) 305-308.

53 J. F. Smith, H. R. Tervit. The successful development of a PRID regime for oestrus synchronization in New Zealand beef cattle. Proc. NZ Soc. Anim. Prod. 4 (1980) 272-279.

54 J. F. Smith, C. C. Kaltenbach. Comparison of techniques for synchronization of oestrus and subsequent fertility in beef cattle. NZ J. Agric. Res. 33 (1990) 449-457.

55 J. F. Smith, L. T. McGowan. Oestrogen and the PRID. Proc. NZ Soc. Anim. Prod. 42 (1982) 87-89.

56 S. Tjondronegoro, P. Williamson, G. J. Sawyer, C. D. Hawkins, S. Atkinson. Effects of progesterone intravaginal devices on oestrus synchronization in post-partum dairy cows. Proc. Aust. Soc. Reprod. Biol. 45 (1986) 45.

57 M. Rosenberg, M. Kaim, Z. Herz, Y. Folman. Comparison of methods for the synchronization of estrus cycles in dairy cows. 1. Effects on plasma progesterone and manifestation of estrus. J. Dairy Sci. 73 (1990) 2807-2816.

58 Y. Folman, M. Kaim, Z. Herz, M. Rosenberg. Comparison of methods for the synchronization of estrus cycles in dairy cows. 2. Effects of progesterone and parity on contraception. J. Dairy Sci. 73 (1990) 2807-2816.

59 A. R. Peters. Calving intervals of beef cows treated with either gonadotrophin releasing hormone or a progesterone releasing device. Vet. Rec. 110 (1982) 515-517.

60 K. J. O'Farrell. Calving rate of dairy cows and heifers following a 12-day progesterone and oestradiol benzoate synchronisation treatment. Ir. J. Agric. Res. 16 (1977) 131-140.

61 G. E. Lamming, D. C. Bulman. The use of milk progesterone radioimmunoassay in the diagnosis and treatment of subfertility in dairy cows. Br. Vet. J. 132 (1976) 507-517.

62 R. L. Fogwell, B. M. Kanyima, A. Villa-Godoy, W. J. Enright, J. J. Ireland. Enhanced precision of estrus and luteinizing hormone after progesterone and prostaglandin in heifers. J. Dairy Sci. 69 (1986) 2179-2185.

63 A. Tegegne, A. C. Warnick, E. Mukasa-Mugerwa, H. Ketema. Fertility of Bos Indicus and Bos Indicus3Bos Taurus crossbreed cattle after estrus synchronization. Theriogenology 31 (1989) 361-370.

64 R. K. Munro. Calving rates or Brahman and Brahman-cross cows to fixed-time insemination after treatment with pregnant mares serum gonadotrophin and intravaginal progesterone. Aust. Vet. J. 65 (1988) 21-23.

65 M. Salaheddine, J. P. Renton, D. N. Logue. Behavioural hormonal and ovarian changes in six multiparous cows synchronized with either Crestar or PRID. Br. Vet. J. 148 (1992) 571-572.

66 M. Alanko, S. Pyorala. The treatment of anoestrus and suboestrus in dairy cattle using a progesterone releasing intravaginal device (PRID) or gonadotrophins. Nord. Vet. Med. 32 (1980) 444-452.

67 S. K. Webel. Control of the estrus cycle in cattle with a progesterone releasing intravaginal device. 8th Int. Cong. Anim. Reprod. Artif. Insem. 3 (1976) 521-522.

68 J. F. Smith, H. R. Tervitt, P. G. Goold. Synchronisation of oestrus and ovulation in beef cows with progesterone releasing intravaginal devices (PRID's): effect of duration of treatment and/or temporary calf removal. Proc. Aust. Soc. Anim. Prod. 13 (1980) 309-312.

69 I. F. Davis, S. R. McPhee, I. J. Clarke. Efficacy of a prostaglandin F 2a analogue or PRID for synchronisation of oestrus in dairy cows. Proc. Aust. Soc. Anim. Prod. 15 (1984) 313-316.

70 R. A. S. Welch, E. Payne, A. D. Hall. Lactogenesis and control of prolactin in cows. Theriogenology 8 (1977) 134.

71 S. M. Lokhande, D. R. Indamdar, B. M. Joshi, M. R. Bhosrekar, P. Humblot, M. Thibier. Progestogen and prostaglandin combined treatments for synchronization of oestrus in post partum crossbred (Bos indicus3Bos taurus) or zebu cows. Rev. Elev. Med. Vet. Pays. Trop. 37 (1984) 73-78.

72 K. L. Macmillan, A. J. Peterson. A new intravaginal progesterone releasing device for cattle (CIDR-B) for oestrous synchronization increasing pregnancy rates and the treatment of post-partum anoestrus. Anim. Reprod. Sci. 33 (1993) 1-25.

73 K. L. Macmillan, V. K. Taufa, D. R. Barnes, A. M. Day. Plasma progesterone concentrations in heifers and cows treated with a new intravaginal device. Anim. Reprod. Sci. 26 (1991) 25-40.

74 K. L. Macmillan, C. R. Burke. Effects of oestrus cycle control on reproductive efficiency. Anim. Prod. Sci. 42 (1996) 307-320.

75 G. A. Bo, G. P. Adams, M. Caccia, M. Martinez, R. A. Pierson, R. J. Mapletoft. Ovarian follicular wave emergence after treatment with progestogen and estradiol in cattle. Anim. Reprod. Sci. 39 (1995) 193-204.

76 D. P. Ryan, S. Snijders, H. Yaakub, K. R. O'Farrell. An evaluation of oestrus synchronization programs in reproductive management of dairy herds. J. Anim. Sci. 73 (1995) 3687-3695.

77 R. K. Munro. Concentrations of plasma progesterone in cows after treatment with 3 types of progesterone pessaries, Aust. Vet. J. 64 (1987) 385-386.

78 S. Tjondronegoro, P. Williamson, G. J. Sawyer, S. Atkinson. Effects of progesterone intravaginal devices on synchronization of estrus in postpartum dairy cows. J. Dairy Sci. 70 (1987) 2162-2167.

79 H. Uehlinger, H. Binder, B. Hauser, P. Rusch, K. Zerobin. TM Hormonanalytischer vergleich der vaginaleinlagen CIDR® and PRID bei ovariektomierten kuhen, Schweiz. Arch. Tierheilk. 137 (1995) 81-86.

80 P. J. Broadbent, L. D. Tregaskes, D. F. Dolman, M. F. Franklin, R. L. Jones. Synchronization of estrus in embryo transfer recipients after using a combination of PRID or CIDR-B plus PGF 2a. Theriogenology 39 (1993) 1055-1065.

81 T. F. Jubb, P. Brightling, J. Malmo, M. T. Larcombe, G. A. Anderson, S. J. Hides. Evaluation of a regimen using a progesterone releasing intravaginal device (CIDR) and PMSG as a treatment for post-partum anoestrus in dairy cattle. Aust. Vet. J. 66 (1989) 334-336.

82 G. F. Duirs, K. L. Macmillan, D. G. McCall, W. H. McMillan, A. M. Day. CIDR systems in suckling beef cows. Proc. Aust. Soc. Reprod. Biol. 19 (1987) 59.

83 K. L. Macmillan, A. M. Day, V. K. Taufa, B. R.. Barnes, T. J. Braggins. Plasma progesterone concentration and oestrus or ovulation in heifers treated with CIDR-type B for at least 7 weeks. Proc. Aust. Soc. Reprod. Biol. 19 (1987) 61.

84 K. L. Macmillan, D. R. Barnes, V. K. Taufa, S. N. Duncan. Plasma progesterone concentrations (PPC) in heifers treated with the CIDR type-B. Proc. Asian/Aust. Assoc. Anim. Prod. 4 (1987) 229.

85 K. L. Macmillan, V. K. Taufa. Effects of using bovine CIDR after first insemination on pregnancy rate and subsequent synchrony. Proc. Asian/Aust. Assoc. Anim. Prod. 4 (1987) 224.

86 J. D. Savio, W. W. Thatcher, G. R. Morris, K. Entwistle, M. Drost, M. R. Mattiacci. Effects of induction of low plasma progesterone concentrations with a progesterone-releasing intravaginal device on follicular turnover and fertility in cattle. J. Reprod. Fertil. 98 (1993) 77-84.

87 J. van Cleeff, M. C. Lucy, C. J. Wilcox, W. W. Thatcher. Plasma and milk progesterone and plasma LH in ovariectomized lactating cows treated with new or used controlled internal release devices. Anim. Reprod. Sci. 27 (1992) 91-106.

88 C. R. Burke, M. Mihm, K. L. Macmillan, J. F. Roche. Some effects of prematurely elevated concentrations of progesterone on luteal and follicular characteristics during the oestrus cycle in heifers. Anim. Reprod. Sci. 35 (1994) 27-39.

89 K. L. Macmillan, S. P. Washburn, H. V. Henderson, S. F. Petch. Effects of varying the progesterone content of CIDR intravaginal devices and multiple CIDR treatments on plasma hormone concentrations and residual hormone content. Proc. NZ Soc. Anim. Prod. 50 (1990) 471-472.

90 Z. Z. Xu, L. J. Burton, K. L. Macmillan. Reproductive performance of synchronised lactating dairy cows. Proc. NZ Soc. Anim. Prod. 55 (1995) 242-244.

91 S. R. McPhee, C. J. Hughes, L. D. Staples, M. B. White, A. H. Williams, I. F. Davis, L. P. Cahill. Synchronisation of oestrus in dairy cows using progesterone administered by controlled internal drug release (CIDR) devices. Proc. Aust. Soc. Anim. Prod. 16 (1976) 103-106.

92 K. L. Macmillan, V. K. Taufa, A. M. Day. Combination treatments for synchronising oestrus in dairy heifers. Proc. NZ Soc. Anim. Prod. 53 (1993) 267-270.

93 C. R. Bunt, M. J. Rathbone, S. Burggraaf, C. Ogle. Development of a QC release assessment method for a physically large veterinary product containing a highly water insoluble drug and the effect of formulation variables upon release. Proc. Int. Symp. Control. Rel. Bioact. Mater. 24 (1997) 145-146.

94 S. Burggraaf, M. J. Rathbone, C. R. Bunt, C. R. Burke, K. L. Pickering. Effect of Shore hardness, inert fillers and progesterone particle size upon the release of progesterone from a controlled release intravaginal drug delivery system. Proc. Int. Symp. Control. Rel. Bioact. Mater. 24 (1997) 147-148.

95 S. McDougall, C. R. Burke, K. L. Macmillan, N. B. Williamson. The effect of pretreatment with progesterone on the oestrus response to oestradiol-17b benzoate in the postpartum dairy cow. Proc. NZ Soc. Anim. Prod. 52 (1992) 157-160.

96 K. L. Macmillan, V. K. Taufa, A. M. Day, S. McDougall. Some effects of using progesterone and oestradiol benzoate to stimulate oestrus and ovulation in dairy cows with anovulatory anoestrus. Proc. NZ Soc. Anim. Prod. 55 (1995) 239-241.

97 Y. Iwazumi, Y. Fukui, R. B. Vargas, C. Nakano, N. Sato, M. Furudate, K. Ohsaki, S. Matsuzaki. Superovulation using CIDR in Holstein cows. J. Reprod. Dev. 40 (1994) 259-266.

98 W. H. McMillan, K. L. Macmillan. CIDR-B for managed reproduction in beef cows and heifers. Proc. NZ Soc. Anim. Prod. 49 (1989) 85-89.

99 R. A. S. Welch. Mating heifers with CIDR. Proc. Ruakura Farmers Conf. 37 (1985) 105-107.

100 A. J. Peterson, H. C. Henderson. Plasma progesterone concentrations in ovariectomized dairy cows treated with a CIDR-B breeding device. J. Reprod. Fertil. Suppl. 43 (1990) 315.

101 S. P. Washburn, H. G. Howard, W. Jochle, K. L. Macmillan. Control of estrus cycles in mature dairy heifers with a progesterone-releasing device. J. Anim. Sci. 67 (1989) 382.

102 D. R. Kerr, M. R. McGowan, C. L. Carroll, F. C. Baldock. Evaluation of three estrus synchronization regimens for use in extensively managed Bos indicus and Bos indicus/taurus heifers in Northern Australia. Theriogenology 36 (1991) 129-141.

103 J. Van Cleeff, K. L. Macmillan, W. W. Thatcher, M. C. Lucy. Estrus synchronization and fertility in heifers with CIDR before and after insemination. J. Anim. Sci. 67 (1989) 383.

104 W. H. McMillan, K. L. Macmillan, A. J. Peterson. Is uterine function compromised in heifers synchronised with a long duration progesterone treatment? Proc. Aust. Soc. Reprod. Biol. 25 (1993) 18.

105 J. Sirois, J. E. Fortune. Lengthening the bovine estrus cycle with low levels of exogenous progesterone: a model for studying ovarian follicular dominance. Endocrinology 127 (1990) 916-925.

106 K. L. Macmillan, V. K. Taufa, A. M. Day. Onset of oestrus and fertility in heifers synchronised with progesterone from a CIDR-Type B for fifteen days. Proc. 11th Int. Cong. Anim. Reprod. 4 (1988) 444.

107 K. L. Macmillan, V. K. Taufa, D. R. Barnes, A. M. Day, R. Henry. Detecting estrus in synchronized heifers using tailpaint and an aerosol raddle. Theriogenology 30 (1988) 1099-1114.

108 N. Ahmad, F. N. Schrick, R. L. Butcher, E. K. Inskeep. Effect of persistent follicles on early embryonic losses in beef cows. Biol. Reprod. 52 (1995) 1129-1135.

109 K. L. Macmillan, V. K. Taufa, A. M. Day, D. R. Barnes. Some effects of administering progesterone per vaginum during metoestrus on oestrous cycle length in heifers. Proc. Aust. Soc. Reprod. Biol. 21 (1989) 105.

110 K. L. Macmillan, A. J. Peterson, D. R. Barnes, S. M. Duncan. Plasma progesterone concentrations in cycling and ovariectomized heifers treated with an intravaginal device (CIDR). Proc. Endocrin. Soc. Aust. 29 (1986) E3.

111 K. L. Macmillan, R. I. Henry, V. K. Taufa, P. Phillips. Calving patterns in seasonal dairy herds. NZ Vet. J. 38 (1990) 151-155.

112 S. C. Cliff, G. R. Morris, I. S. Hook, K. L. Macmillan. Calving patterns in dairy heifers following single set-time inseminations and re-synchrony preceding second inseminations. Proc. NZ Soc. Anim. Prod. 55 (1995) 70-71.

113 K. L. Macmillan, V. K. Taufa, Pregnancy rates to first insemination in dairy cattle previously treated with progesterone during late dioestrus and with or without oestradiol benzoate, Proc. Aust. Soc. Reprod. Biol. 25 (1993) 75.

114 K. L. Macmillan, V. K. Taufa, C. R. Burke. Effects of oestradiol on synchrony patterns in cattle treated with an intravaginal progesterone device. Proc. Aust. Soc. Reprod. Biol. 25 (1993) 94.

115 S. McDougall, K. L. Macmillan, N. B. Williamson. Effect of treatment of non-cycling lactating dairy cows with progesterone and/or oestradiol. Proc. Aust. Soc. Reprod. Biol. 25 (1993) 96.

116 C. R. Burke, K. L. Macmillan, M. P. Boland. Oestradiol potentiates a prolonged progesterone-induced suppression of LH release in ovariectomised cows. Anim. Reprod. Sci. 45 (1996) 13-28.

117 A. R. Dick, K. L. Macmillan. Variation in patterns of oestrus behaviour associated with the timing of an intravaginal device removal in dairy heifers. Proc. 13th Int. Cong. Anim. Reprod. 3 (1996) P19-4.

118 P. R. Lynch, K. L. Macmillan. Strategic use of an intravaginal device to synchronise returns-to-oestrus and to allow early pregnancy testing in recipient heifers. Proc. NZ Embryo Transfer Workshop, Hamilton, 1994, pp. 32-33.

119 C. R. Burke, K. L. Macmillan. Diurnal variations in circulating progesterone in ovariectomised cows treated with an intravaginal progesterone device. Proc. Aust. Soc. Reprod. Biol. 27 (1995) 41.

120 K. L. Macmillan, V. K. Taufa, D. R. Barnes, A. M. Day. Oestrus detection in synchronised heifers. Proc. 11th Int. Cong. Anim. Reprod. 4 (1988) 443.

121 A. M. Day, K. L. Macmillan, W. J. Killen, C. Burke, C. H. Mirams. The use of a CIDR-B device with an oestrogen capsule in an artificial breeding programme with suckling beef cows. Proc. Aust. Assoc. Anim. Artif. Breed. 36 (1991) 4.

122 K. L. Macmillan, J. G. E. Pickering. Using CIDR-Type B and PMSG to treat anoestrum in New Zealand dairy cows. Proc. 11th Int. Cong. Anim. Reprod. 4 (1988) 442.

123 C. R. Burke, K. L. Macmillan. The effects of intravaginal administration of progesterone and oestradiol benzoate on circulating LH in ovariectomised cows. Proc. Aust. Soc. Reprod. Biol. 27 (1995) 38.

124 K. L. Macmillan, V. K. Taufa, G. R. Morris. Patterns of oestrus vary with different forms of synchrony. Proc. NZ Soc. Anim. Prod. 56 (1996) 347-349.

125 K. L. Macmillan, V. K. Taufa, A. M. Day. Varying the form of oestradiol administration in anoestrous cows previously treated with progesterone. Proc. NZ Soc. Anim. Prod. 56 (1996) 350-352.

126 A. R. Dick, K. L. Macmillan, V. K. Taufa, R. S. Morris. Some effects of controlling the variation in return to service intervals in dairy cows with progesterone from an intravaginal device and on cow-side pregnancy testing. Proc. 12th Int. Cong. Anim. Reprod. 3 (1992) 1118-1120.

127 S. McDougall, K. L. Macmillan, N. B. Williamson. The effect of progesterone pre-treatment on luteal junction following oestradiol treatment in the non-cycling post partum dairy cow. Proc. NZ Soc. Endocrin., 1992, Abstract 39.

128 K. L. Macmillan, V. K. Taufa, D. R. Barnes, A. M. Day. Use of CIDR-Type B for oestrus cycle control in maiden dairy heifers. Proc. Asian/Aust. Assoc. Anim. Prod. 4 (1987) 220.

129 K. L. Macmillan, V. K. Taufa, A. M. Day, A. J. Peterson. Effects on supplemental progesterone on pregnancy rates in cattle. J. Reprod. Fertil. 43 (1990) 304.

130 C. R. Burke, S. Burggraaf, C. R. Bunt, M. J. Rathbone, K. L. Macmillan. Use of pregnant dairy cows in product development of the intravaginal progesterone releasing (CIDR) device. Proc. NZ Soc. Anim. Prod., 57 (1997).

131 L. V. Swanson, B. W. Wickham, K. L. Macmillan. Effect of exogenous progesterone (P4) on follicular waves in dairy beef heifers. J. Dairy Sci. 73 (1990) 177.

132 J. Sreenan. Retention of intravaginal sponge-pessaries by cattle. Vet. Rec. 94 (1974) 45-47.

133 S. R. Davis, R. A. S. Welch, M. G. Pierce, A. J. Peterson. Induction of lactation in nonpregnant cows by oestradiol 17b and progesterone from an intravaginal sponge. J. Dairy Sci. 66 (1983) 450-457.

134 P. F. Scanlon, W. J. Neville, T. G. Burgess, J. W. Macpherson. Synchronisation of oestrus in cattle by intravaginal application of progesterone with oestrogen administration. Can. J. Anim. Sci. 51 (1971) 250-251.

135 P. F. Scanlon, B. Sreenan, I. Gordon. Synchronization of oestrus in heifers by intravaginal application of progesterone. Vet. Rec. 90 (1972) 440-441.

136 P. G. Hignett, H. Boyd, D. F. Wishart. Syncronisation of oestrus in Ayrshire heifers by the use of progestinated intravaginal pessaries. Vet. Rec. 86 (1970) 528-531.

137 J. F. Smith. Synchronisation of oestrus in cattle. NZ J. Agric. August (1974) 26-30.

138 M. J. Carrick, J. N. Shelton. The synchronisation of oestrus in cattle with progestogen impregnated intravaginal sponges. J. Reprod. Fertil. 14 (1967) 21-32.

139 D. F. Wishart, B. D. Hoskin. Synchronization of oestrus in heifers using intra-vaginal pessaries impregnated with SC-9880 and PMSG. J. Reprod. Fertil. 17 (1968) 285-289.

140 H. Shimizu, Y. Toyoda, S. Takeuchi, T. Kawai, S. Adachi. Synchronisation of oestrus and subsequent fertility of beef cattle following the intravaginal administration of gestagen. J. Reprod. Fertil. 13 (1967) 555-558.

141 R. W. Moore, J. F. Smith. Effect of progestogen intravaginal sponges and PMSG on synchronization of oestrus in maiden heifers and on interval from calving to oestrus in beef cows. NZ J. Expt. Agric. 8 (1980) 199-203.

142 D. H. Hale, R. B. Symington. Control of sexual activity in ranch cows by intramuscular and intravaginal administration of progestogens. J. Reprod. Fertil. 18 (1969) 193-199.

143 J. F. Smith, R. J. Fairclough, A. J. Peterson. Plasma levels of progesterone provera oestradiol-17b and 13,14 dihydro-15-keto-prostaglandin F in cows treated with Provera-impregnated intravaginal sponges. J. Reprod. Fertil. 55 (1979) 359-364.

144 Th. Hornykiewytsch. Intra-vaginal application system (INVAS) for controlled drug release in animals. Acta Pharm. Technol. 34 (1988) 68.

145 D. Hiller. Th. Hornykiewytsch. Process for preparing an intravaginal application system. U.S. Pat. No. 5,398,698 (1995).

146 J. F. Roche. Retention rate in cows and heifers of intravaginal silastic coils impregnated with progesterone. J. Reprod. Fertil. 46 (1976) 253-255.

147 R. Rajamahendran, P. C. Lague, R. D. Baker. Serum hormone levels and occurrence of oestrus following use of an intravaginal device containing progesterone and oestradiol 17b in heifers. Anim. Reprod. Sci. 3 (1980) 271-277.

148 R. Rajamahendran, P. C. Lague, R. D. Baker. Estrus and LH release in ovariectomized heifers following vaginal devices containing ovarian steroids. J. Anim. Sci. 49 (1979) 554-559.

149 R. Rajamahendran, L. Forgrave, P. C. Lague, R. D. Baker. Control of estrus in heifers with intravaginal progesterone devices. Can. J. Anim. Sci. 55 (1975) 787.

150 ONSETT technical specifications. Advanced Animal Technology Limited. New Zeeland. www.aat.co.nz

151 P. F. Scanlon, T. D. Burgess. Subcutaneous and oral applications of progestogens for control of estrus in heifers. Can. J. Anim. Sci. 51 (1971) 540-541.

152 J. N. Wiltbank, J. C. Sturges, D. Wideman, D. G. LeFever, L. C. Faulkner. Control of estrus and ovulation using subcutaneous implants and estrogens in beef cattle. J. Anim. Sci. 33 (1971) 600-606.

153 S. E. Curl, W. Durfey, R. Patterson, D. W. Zinn. Synchronization of estrus in cattle with subcutaneous implants. J. Anim. Sci. 27 (1968) 1189.

154 C. O. Woody, R. A. Pierce, Influence of day of estrus cycle at treatment on response to estrus cycle regulation by norethanrolone implants and estradiol valerate injections, J. Anim. Sci. 39 (1974) 903-906.

155 R. E. Short, R. A. Bellows, J. B. Carr, R. B. Staigmiller, R. D. Randel. Induced or synchronized puberty in heifers. J. Anim. Sci. 43 (1976) 1254-1258.

156 J. F. Roche. Effect of short-term progesterone treatment on estrus response and fertility in heifers. J. Reprod. Fertil. 40 (1974) 433-440.

157 J. F. Roche, Synchronisation of oestrus in heifers with implants of progesterone. J. Reprod. Fertil. 41 (1974) 337-344.

158 J. F. Roche, J. P. Crowley. The long-term suppression of heat in cattle with implants of melengestrol acetate. Anim. Prod. 16 (1973) 245-250.

159 J. F. Roche, J. P. Crowley, The use of implants containing steroids for growth promotion and control of oestrus in cattle. Anim. Prod. 13 (1971) 385.

160 Y. W. Chien, E. P. K. Lau. Controlled drug release from polymeric delivery devices IV. In vitro-in vivo correlation of subcutaneous release of norgestomet from hydrophilic implants. J. Pharm. Sci. 65 (1976) 488-492.

161 D. J. Kesler, R. J. Favero, T. R. Troxel. A comparison of Hydron and silicone implants in the bovine norgestomet and estradiol valerate estrus synchronization procedure. Drug Dev. Ind. Pharm. 21 (1995) 475-485.

162 D. J. Kesler, R. J. Favero, In vitro and in vivo secretion of norgestomet from matrix silicone and Hydron implants and pregnancy rates of females synchronized with norgestomet. Proc. Int. Symp. Control. Rel. Bioact. Mater. 16 (1989) 435-436.

163 J. C. Spitzer, W. C. Burrell, D. G. LeFever, R. W. Whitman, J. N. Wiltbank. Synchronization of estrus in beef cattle. 1. Utilization of a norgestomet implant and injection of estradiol valerate. Theriogenology 10 (1978) 181-200.

164 W. M. Moseley, D. W. Forrest, C. C. Kaltenbach, T. G. Dunn. Effect of norgestomet on peripheral levels of progesterone and estradiol-17b in beef cows. Theriogenology 11 (1979) 331-341.

165 D. L. Hixon, D. J. Kesler, T. R. Troxel, D. L. Vincent, B. S. Wiseman. Reproductive hormone secretions in first service conception rate subsequent to ovulation control with SYNCRO-MATE-B. Theriogenology 16 (1981) 219-229.

166 R. Rajamahendran, C. Taylor. Follicular dynamics and temporal relationships among body temperature, oestrus the surge of luteinising hormone and ovulation in Holstein heifers treated with norgestomet. J. Reprod. Fertil. 92 (1991) 461-467.

167 P. D. Burns, J. C. Spitzer, W. C. Bridges, D. M. Henricks, B. B. Plyler. Effects of metestrus administration of a norgestomet implant and injection of norgestomet and estradiol valerate on luteinizing hormone release and development and function of corpora lutea in suckled beef cows. J. Anim. Sci. 71 (1993) 983-988.

168 Y. W. Chien. Controlled administration of estrus-synchronizing agents in livestock, in Novel Drug Delivery Systems Fundamentals Developmental Concepts Biomedical Assessments. Marcel Dekker, New York, 1982, pp. 413-463.

169 R. Duncan, L. W. Seymour. Controlled Release Technologies—A Survey of Research and Commercial Applications. Elsevier Science Publishers. London, 1989, p. 18.

170 J. W. Lauderdale, C. W. Kasson, M. L. Ogilvie. Infection of implant sites. J. Anim. Sci. 35 (1972) 246.

171 M. Salaheddine, J. P. Renton, D. N. Logue. Behavioural hormonal and ovarian changes in six multiparous cows synchronized with either Crestar or PRID. Br. Vet. J. 148 (1992) 571-572.

172 K. L. Macmillan, V. K. Taufa, G. R. Morris. Patterns of oestrus vary with different forms of synchrony. Proc. NZ Soc. Anim. Prod. 56 (1996) 347-349.

173 B. G. Lowman, N. A. Scott, P. R. Scott. An evaluation of some breeding management options in beef herds in the United Kingdom. Vet. Rec. 135 (1994) 9-12.

174 L. D. Tregaskes, P. J. Broadbent, D. F. Dolman, S. P. Grimmer, M. F. Franklin. Evaluation of Crestar a synthetic progestogen regime for synchronizing oestrus in maiden heifers used as recipients of embryo transfers. Vet. Rec. 134 (1994) 92-94.

175 S. G. Revell. Heterospermic insemination of cattle using three bulls of the same breed. Vet. Rec. 133 (1993) 20.

176 V. K. Sharma, R. C. Gutpa, N. Khurana, S. K. Khar. Oestrus synchronization and superovulation using equine FSH in crossbred ewes. Vet. Rec. 135 (1994) 164-165.

177 D. N. Logue, M. Salaheddine, J. P. Renton. A comparison of two techniques for the synchronisation of oestrus in dairy heifers. Vet. Rec. 129 (1991) 171-173.

178 R. S. Bezwada. Liquid copolymers of epsilon-caprolactone and lactide. U.S. Pat. No. 5,442,033, 15 Aug. 1995.

179 R. S. Bezwada, S. C. Arnold. Liquid absorbable copolymers for parenteral applications. U.S. Pat. No. 5,653,992, 5 Aug. 1997.

180 P. C. Painter. M. M. Coleman (Eds.). Fundamentals of Polymer Science, Technomic, Lancaster, Pa., 1994, pp. 4-5.

181 S. Einmahl, F. Behar-Cohen, C. Tabatabay, M. Savoldelli, F. D. Hermies, D. Chauvaud, J. Heller, R. Gurny. A viscous bioerodible poly(ortho ester) as a new biomaterial for intraocular application. J. Biomed. Mater. Res. 50 (2000) 566-577.

182 K. A. Walter, M. A. Cahan, A. Gur, B. Tyler, J. Hilton, O. M. Colvin, P. C. Burger, A. Domb, H. Brem. Interstitial Taxol delivered from a biodegradable polymer implant against experimental malignant glioma. Cancer Res. 54 (1994) 2207-2212.

183 X. Zhang, J. K. Jackson, W. Wong, W. Min, T. Cruz, W. L. Hunter, H. M. Burt. Development of biodegradable polymeric paste formulations for taxol: an in-vivo and in-vitro study. Int. J. Pharm. 137 (1996) 199-208.

184 F. Liu, B. C. Wilson. Hyperthermia and photodynamic therapy, in: I. Tannock, R. P. Hill (Eds.), Basic Science of Oncology, McGraw-Hill, New York, 1998, pp. 443-453.

185 S. K. Dordanoo, A. M. C. Oktaba, W. Hunter, W. Min, T. Cruz, H. M. Burt. Release of Taxol from poly caprolactone pastes: effect of water soluble additives. J. Control. Release 44 (1997) 87-94.

186 C. G. Pitt. Poly-e-caprolactone and its copolymers, in: M. Chasin, R. Langer (Eds.), Biodegradable Polymers as Drug Delivery Systems, Marcel Decker, New York, 1990, pp. 71-120.

187 C. I. Winternitz, J. K. Jackson, J. K. Jackson, A. M. Oktaba, H. M. Burt. Development of a polymeric surgical paste formulation for taxol. Pharm. Res. 13 (3) (1996) 368-375.

188 J. K. Jackson, M. E. Gleave, V. Yago, E. Beraldi, W. L. Hunter, H. M. Burt. The suppression of human prostate tumor growth in mice by the intratumoral injection of a slow-release polymeric paste formulation of Paclitaxel. Cancer Res. 60 (2000) 4146-4151.

189 P. A. Davis, S. Cousins. Biodegradable injectable drug delivery polymer. U.S. Pat. No. 5,384,333, 24 Jan. 1995.

190 P. C. Painter. M. M. Coleman (Eds.), Fundamentals of Polymer Science, Technomic, Lancaster, Pa., 1994, pp. 4-5.

191 R. L. Dunn, J. P. English, D. R. Cowsar, D. D. Vanderbelt. Biodegradable in-situ forming implants and methods of producing the same. U.S. Pat. No. 5 278 201, 11 Jan. 1994.

192 R. L. Dunn, J. P. English, D. R. Cowsar, D. D. Vanderbelt. Biodegradable in-situ forming implants and methods of producing the same. U.S. Pat. No. 5 278 202, 11 Jan. 1994.

193 S. J. Peter, J. A. Nolley, M. S. Widmer, J. E. Merwin, M. J. Yaszemski, A. W. Yasko, P. S. Engel, A. G. Mikos. In-vitro degradation of a poly(propylene fumarate)/b-tricalcium phosphate composite orthopedic scaffold. Tissue Eng. 3 (1997) 207-215.

194 A. K. Burkoth, K. S. Anseth. A review of photocrosslinked polyanhydrides: in-situ forming degradable networks. Biomaterials 21 (2000) 2395-2404.

195 R. L. Dunn, J. P. English, D. R. Cowsar, D. D. Vanderbelt. Biodegradable in-situ forming implants and methods for producing the same. U.S. Pat. No. 5,340,849, 23 Aug. 1994.

196 L. A. Moore, R. L. Norton, S. L. Whitman, R. L. Dunn. An injectable biodegradable drug delivery system based on acrylic terminated poly e-caprolactone. presented at the 21st Annual Meeting of the Society of Biomaterials, CA, 1995.

197 L. A. Moore, R. L. Norton, S. L. Whitman, R. L. Dunn. An injectable biodegradable drug delivery system based on acrylic terminated poly e-caprolactone. presented at the 21st Annual Meeting of the Society of Biomaterials, CA, 1995.

198 G. Sund, J. Rosequist. Morphological changes in bone following implantation of methyl methacrylate. Acta Orthop. Scand. 54 (1983) 148-156.

199 J. S. Temenoff, A. G. Mikos. Injectable biodegradable materials for orthopedic tissue engineering. Biomaterials 21 (2000) 2405-2412.

200 G. Biehl, U. Hanser, J. Harms. Experimental studies on heat development in bone during polymerization of bone cement. Intra-operative measurement of temperature in normal blood circulation and in bloodlessness. Arch. Orthop. Unfallchir. 78 (1974) 62-69.

201 M. A. Saleem, S. I. Ahmed. Tephrosia purpurea ameliorates benzoyl peroxide-induced coetaneous toxicity in mice: diminution of oxidative stress. Pharm. Pharmacol. Commun. 5 (7) (1999) 455-461.

202 I. B. Gimenez-Conti, R. L. Binder, D. Johnston, T. J. Slaga. Comparison of the skin tumor-promoting potential of different organic peroxides in SENCAR mice. Toxicol. Appl. Pharmacol. 149 (1) (1998) 73-79.

203 J. L. Zhao, Y. Sharma, M. L. Chatterjee, R. Agarwal. Inhibitory effect of a falvonoid antioxidant silymarin on benzoyl peroxide-induced tumor promotion, oxidative stress and inflammatory response in SENCAR mouse skin. Carcinogenesis 21 (4) (2000) 811-816.

204 T. X. Viegas, L. E. Reeve, R. L. Henry. Medical uses of in-situ formed gels. U.S. Pat. No. 5,318,780, 7 Jun. 1994.

205 H. Cui, P. B. Messermith. Thermally triggered gelation of alginate for controlled release. in: I. McCulloch, S. W. Shalaby (Eds.), Tailored Polymeric Materials For Controlled Delivery Systems, American Chemical Society, Washington, D.C., 1998, pp. 203-211.

206 D. Papahadjopoulos, K. Jacobsen, S. Nir, T. Isac. Phase transitions in phospholipid vesicles, Biochim. Biophys. Acta 311 (1973) 330.

207 D. Marsh, A. Watts, P. F. Knowles. Cooperativity of phase transition in single bilayer and multi bilayer lipid vesicles. Biochim. Biophys. Acta 465 (3) (1977) 500-514.

208 P. G. Kibat, Y. Igari, M. A. Wheatley, H. N. Eisen, R. Langer. Enzymatically activated microencapsulated liposomes can provide pulsatile drug release. FASEB J. 4 (1990) 2533-2539.

209 W. A. Soskolne. Subgingival delivery of therapeutic agents in the treatment of periodontal diseases. Crit. Rev. Oral Biol. Med. 8 (1997) 164-174.

210 E. Westhaus, P. B. Messermith. Triggered release of calcium from lipid vesicles: a bioinspired strategy for rapid gelation of polysaccharide and protein hydrogels. Biomaterials 22 (2001) 453-462.

211 S. Cohen, E. Lobel, A. Trevgoda, Y. Peled. A novel in-situ forming drug delivery system from alginates undergoing gelation in the eye. J. Control. Release 44 (1997) 201-208.

212 Y. Suzuki, Y. Nishimura, M. Tanihara, K. Suzuki, Y. Shimizu, Y. Yamawaki, Y. Kakimaru. Evaluation of a novel alginate gel dressing: cytotoxicity to fibroblasts in vitro and foreign-body reaction in pig's skin in vivo. J. Biomed. Mater. Res. 39 (2) (1998) 317-322.

213 A. B. G. Lansdown, M. J. Payne. An evaluation of the local reaction and biodegradation of calcium sodium alginate (kaltostat) following subcutaneous implantation in the rat, J. R. Coll. Surg. Edinb. 39 (1994) 284-288.

214 N. H. Shah, A. S. Railkar, F. C. Chen, R. Tarantino, S. Kumar, M. Murjani, D. Palmer, M. H. Infeld, A. W. Malick. A biodegradable injectable implant for delivering micro and macromolecules using poly(lactic-co-glycolic)acid copolymers. J. Control. Release 27 (1993) 139-147.

215 R. E. Eliaz, J. Kost. Characterization of a polymeric PLGA-injectable implant delivery system for the controlled release of proteins. J. Biomed. Mater. Res. 50 (2000) 388-396.

216 B. Jeong, Y. H. Bae, S. W. Kim. In-situ gelation of PEG-PLGA-PEG triblock copolymer aqueous solutions and degradation thereof. J. Biomed. Mater. Res. 50 (2000) 171-177.

217 A. Paavola, J. Yliruusi, P. Rosenberg. Controlled release and dura mater permeability of lidocaine and ibuprofen from injectable poloxamer-based gels. J. Control. Release 52 (1-2) (1998) 169-178.

218 R. A. Siegel, B. A. Firestone. pH-dependent equilibrium swelling properties of hydrophobic poly-electrolyte copolymer gels. Macromolecules 21 (1988) 3254-3259.

219 T. Tanaka. Phase transitions in ionic gels. Phys. Rev. Lett. 45 (1980) 1636-1639.

220 R. L. Dunn, J. P. English, D. R. Cowsar, D. P. Vanderbelt. Biodegradable in-situ forming implants and methods of producing the same. U.S. Pat. No. 4,938,763, 3 Jul. 1990.

221 R. Dunn, G. Hardee, A. Polson, A. Bennett, S. Martin, R. Wardley, W. Moseley, N. Krinick, T. Foster, K. Frank, S. Cox. In-situ forming biodegradable implants for controlled release veterinary applications. Controlled Release Society, Inc. Proc. Intern. Symp. Rel. Bioact. Mater., 1995, 22.

222 W. J. Lambert, K. D. Peck. Development of an in situ forming biodegradable poly-lactide-co-glycolide system for the controlled release of proteins. J. Control. Release 33 (1995) 189-195.

223 E. G. Duysen, S. L. Whitman, N. L. Krinick, S. M. Fujita, G. L. Yewey. An injectable, biodegradable delivery system for antineoplastic agents. AAPS, Pharm. Res., American Association Of Pharmaceutical Scientists, presentation 7575, 1994.

224 J. M. Sherman, S. M. Fujita. Localized delivery of bupivacaine from Atrigel formulations for the management of postoperative pain. AAPS, Pharm. Res., American Association Of Pharmaceutical Scientists, presentation 7574, 1994.

225 R. L. Dunn, G. L. Yewey, E. G. Duysen, K. R. Frank, W. E. Huffer, R. Pieters. Bone regeneration with the Atrigel polymer system, presented at the Portland Bone Symposium, Portland, Oreg., 1995.

226 K. R. Frank, E. G. Duysen, G. L. Yewey, W. E. Huffer, R. Pieters. Controlled release of bioactive growth factors from a biodegradable delivery system. Pharm. Res., American Association Of Pharmaceutical Scientists, presentation 2070, 1994.

227 B. K. Lowe, R. L. Norton, E. L. Keeler, K. R. Frank, A. J. Tipton. The effects of gamma irradiation on poly D,L-lactide as a solid and in N-methyl 2-pyrrolidone solutions, presented at the 19th Annual Meeting of the Society of Biomaterials, AL, 1993.

228 R. L. Dunn, A. J. Tipton, G. L. Southard, J. A. Rogers. Biodegradable polymer composition. U.S. Pat. No. 5,599,552, 4 Feb. 1997.

229 M. L. Radomsky, G. Brouwer, B. J. Floy, D. J. Loury, F. Chu, A. J. Tipton, L. M. Sanders. The controlled release of Ganirelix from the Atrigel injectable implant system. Proc Intern. Symp. Control. Rel. Bioact. Mater. 20, 1993.

230 M. L. Shively, A. T. Bennett, B. A. Coonts, W. D. Renner, J. L. Southard. Physico-chemical characterization of polymeric injectable implant delivery system. J. Control. Release 33 (1995) 237-243.

231 B. L. Chandrashekar, M. Zhou, E. M. Jarr, R. L. Dunn. Controlled release liquid delivery compositions with low initial drug burst. U.S. Pat. No. 6,143,314, 2000.

232 M. A. Royals, S. M. Fujuta, G. L. Yewey, J. Rodriguez, P. C. Schultheiss, R. L. Dunn. Biocompatibility of a biodegradable in-situ forming implant system in rhesus monkeys. J. Biomed. Mater. Res. 45 (1999) 231-239.

233 H. Kranz, G. A. Brazeau, J. Napaporn, R. L. Martin, W. Millard, R. Bodmeier. Myotoxicity studies of injectable biodegradable in-situ forming drug delivery systems. Int. J. Pharm. 212 (2001) 11-18.

234 G. Chandrashekar, N. Udupa. Biodegradable injectable implant systems for long term drug delivery using poly-(lactic-co-glycolic) acid copolymers. J. Pharm. Pharmacol. 48 (1996) 669-674.

235 U. V. Singh, N. Udupa, R. Kamath, P. Umadevi. Enhanced antitumor efficacy of methoteraxate poly(lactic-co-glycolic) acid injectable gel implants in mice bearing sarcoma-180. Pharm. Sci. 3 (1997) 133-136.

236 R. Cherng-Chyi Fu, D. M. Lidgate, J. L. Whatley, T. McCullough. The biocompatibility of parenteral vehicles in vivo/in vitro screening comparison and the effect of excipients on haemolysis. J. Parent. Sci. Technol. 41(5):164-168.

237 N. J. Medlicott, K. A. Foster, K. L. Audus, S. Gupta, V. J. Stella. Comparison of the effects of potential parenteral vehicles for poorly water soluble anticancer drugs. J. Pharm. Sci. 87 (9) (1998) 1138-1143.

238 R. E. Eliaz, D. Wallach, J. Kost. Delivery of soluble tumor necrosis factor receptor from in-situ forming PLGA implants: in-vivo. Pharm. Res. 17 (12) (2000) 1546-1550.

239 A. H. Kibbe, A. Wade, P. J. Weller. HandBook of Pharmaceutical Excipients, Am. Pharm. Assoc. and Pharm. Soc. GB, 1994.

240 M. A. Crowther, A. Pilling, K. Owen. The evaluation of glycofurol as a vehicle for use in toxicity studies. Hum. Exp. Toxicol. 16 (1997) 406.

241 A. J. Spiegel, M. M. Noseworthy. Use of non-aqueous solvents in parenteral products. J. Pharm. Sci. 52 (1963) 917-926.

242 V. R. Budden, U. G. Kuhl, G. Buschmann. Studies on pharmacodynamic activity of several drug solvents. Arzneim.-Forsch. Drug Res. 28 (1978) 1571-1579.

243 B. O. Haglund, J. Rajashree, K. J. Himmelstein. An in-situ gelling system for parenteral delivery. J. Control. Release 41 (1996) 229-235.

244 F. A. Ismail, J. Napaporn, J. A. Hughes, G. A. Brazeau. In situ gel formulations for gene delivery: release and myotoxicity studies. Pharm. Dev. Technol. 5 (2000) 391-397.

245 T. O'Hara, A. Dunne, J. Butler, J. Devane. A review of methods used to compare dissolution profile data. PSST 1 (5) (1998) 214-223.

246 R. A. Jain, C. T. Rhodes, A. M. Railkar, A. W. Malick, N. A. Shah. Controlled release of drugs from injectable in situ formed biodegradable PLGA microspheres: effect of various formulation variables. Eur. J. Pharm. Biopharm. 50 (2000) 257-262.

247 D. A. Smith, A. J. Tipton. A novel parenteral delivery system. Pharm. Res. 13 (9) (1996) 300.

248 [115] P. J. Burns, J. W. Gibson, A. J. Tipton. Compositions suitable for controlled release of the hormone GNRH and its analogs. U.S. Pat. No. 6,051,558, 18 Apr. 2000.

249 A. J. Tipton. High viscosity liquid controlled delivery system as a device. U.S. Pat. No. 5,968,542, 19 Oct. 1999.

250 S. Benita. Microencapsulation, Methods and Industrial Applications. Marcel Dekker Inc., New York 1996; vol 73; pp 587-632.

251 M. Frangione-Beebe, R. T. Rose, P. T. Kaumaya, S. P. Schwendeman. J. Microencap. 18 (2001) 663-677.

252 A Esquisabel, R. Hernandez, M. Iguartua. J. Microencap. 14 (1997) 627-638.

253 D. Poncelet. Production of alginate beads by emulsification internal gelation. Ann. New York Acad. Sci. 2001; vol.944: 74-82.

254 B. Heurtault, P. Saulnier, B. Pech, J. E. Proust, J. P. Benoit. Pharma. Res. 19 (2001) 21-31.

255 A. Lamprecht, U. F. Schafer, C. M. Lehr. Eur. J. Pharma. Biopharma. 49 (2000) 1-9.

256 A. Bachtsi, C. Boutris, C. Kiparissides. J. Appl. Polym. Sci. 60 (1996) 9-20.

257 A. R. Kulkarni, K. S. Soppimath, T. M. Aminabhavi, A. M. Dave, M. H. Mchat. J. Control. Rel. 63 (2000) 97-105.

258 M. C. Levy, M. C. Andry. J. Microencap. 8 (1991) 335-347.

259 S. Abrahan, R. Vieth, J. Diane. Pharma. Dev. Technol. 1 (1996) 63-68.

260 B. F. Gibbs, S. Kermasha, I. Alli, C. N. Mulligan. Int. J. Food Sci. Nutr. 50 (1999) 213-224.

261 Y. Senuma, C. Lowe, Y. Zweiffel, J. G. Hilborn, I. Marison. Biotechnol. Bioeng. 67 (2000) 616-622.

262 J. C. Ogbonna, M. Matsumura, T. Yamagata, H. Sakuma, H. J. Kataoka. Ferment. Bioeng. 68 (1989) 40-48.

263 U. Prusse, F. Bruske, J. Breford, K. D. Vorlop. Chem, Eng. Tech. 21 (1998) 153-157.

264 B. Bugarski, Q. L. Li, M. F. A. Goosen, D. Poncelet, R. J. Neufeld, G. Vunjak. AICHE J. 40 (1994) 1026-1031.

265 J. P. Halle, F. A. Leblond, J. F. Pariseau, P. Jutras, M. J. Brabant, Y. Lepage. Cell Transplantation. 3 (1994) 365-372.

266 A. C. Hulst, J. Tramper, K. Vantriet, J. M. M. Westerbeek. Biotech. Bioeng. 27 (1985) 870-876.

267 W. M. Obeidat. Recent patents review in microencapsulation of pharmaceuticals using the emulsion solvent removal methods. Recent Patents on Drug Delivery and Formulation. 3 (2009) 178-192.

268 D. Ray, P. S. Gils, G. P. Mohanta, R. Manavalan, P. K. Sahoo. Comparative delivery of diltiazem hydrochloride through synthesized polymer: Hydrogel and hydrogel microspheres. J. Appl. Polym. Sci. 116 (2010) 959-968.

269 A. G. Sullad, L. S. Manjeshwar, T. M. Aminabhavi.Controlled release of theophylline from interpenetrating blend microspheres of poly(vinyl alcohol) and methyl cellulose. J. Appl. Polym. Sci. 116 (2010) 1226-1235.

270 X. Bai, Z. F. Ye, Y. Z. Qu, Y. F. Li, Z. Wang. Immobilization of nanoscale Fe0 in and on PVA microspheres for nitrobenzene reduction. J. Hazardous Mat. 172 (2009) 1357-1364.

271 L. C. Wang, H. Wu, X. G. Chen, L. D. Li, Q. X. Ji, C. S. Liu, L. J. Yu, Q. S. Zhao. Biological evaluation of a novel chitosan-PVA-based local delivery system for treatment of periodontitis. J. Biomed. Mat. Res. Part A 91 (2009) 1065-1076.

272 R. C. Mundargi, S. A. Patil, P. V. Kulkarni, N. N. Mallikarjuna, T. M. Aminabhavi. Sequential interpenetrating polymer network hydrogel microspheres of poly(methacrylic acid) and poly(vinyl alcohol) for oral controlled drug delivery to intestine. J. Microencap. 25 (2008) 228-240.

273 X. Cui, H. M. Zhang, M. S. Ye, F. L. Yang. Preparation and characterization of cross-linked polyvinyl alcohol microspheres as a novel precoating reagent in dynamic membrane. Environm. Sci. 29 (2008) 960-965.

274 H. M. Jung, E. M. Lee, B. C. Ji, Y. Deng, J. D. Yun, J. H. Yeum. Poly(vinyl acetate)/poly(vinyl alcohol)/montmorillonite nanocomposite microspheres prepared by suspension polymerization and saponification. Coll. Polym. Sci. 285 (2007) 705-710.

275 M. D. Kurkuri, T. M. Aminabhavi. Poly(vinyl alcohol) and poly(acrylic acid) sequential interpenetrating network pH-sensitive microspheres for the delivery of diclofenac sodium to the intestine. J. Controlled Release 96 (2004) 9-20.

276 S. G. Lee, W. S. Lyoo. Preparation of monodisperse poly(vinyl alcohol) microspheres by heterogeneous surface saponification and iodine complex formation. J. Appl. Polym. Sci. 107 (2008) 1701-1709.

277 M. Constantin, G. Fundueanu, F. Bortolotti, R. Cortesi, P. Ascenzi, E. Menegatti. A novel multicompartimental system based on aminated poly(vinyl alcohol) microspheres/succinoylated pullulan microspheres for oral delivery of anionic drugs. Int. J. Pharm. 330 (2007). 129-137.

278 A. A. A. De Queiroz, E. D. Passes, S. De Brito Alves, G. S. Silva, O. Z. Higa, M. Vitolo. Alginate-poly(vinyl alcohol) core-shell microspheres for lipase immobilization. J. Appl. Polym. Sci. 102 (2006) 1553-1560.

279 B. C. Thanoo, M. C. Sunny, A. Jayakrishnan. Controlled release of oral drugs from cross-linked polyvinyl alcohol microspheres. J. Pharmaceu. Pharmacol. 45 (1993) 16-20.

280 T. Y. Ting, I. Gonda, E. M. Gipps. Microparticles of polyvinyl alcohol for nasal delivery. I. Generation by spray-drying and spray-desolvation. Pharm. Res. 9 (1992) 1330-1335.

281 C. Brochhausen, R. Zehbe, B. Watzer, S. Halstenberg, F. Gabler, H. Schubert, C. J. Kirkpatrick. Immobilization and controlled release of prostaglandin E2 from poly-L-lactide-co-glycolide microspheres. J. Biomed. Mat. Res. Part A 91 (2009) 454-462.

282 S. Puthli, P. Vavia. Formulation and performance characterization of radio-sterilized “Progestin-only” microparticles intended for contraception. AAPS Pharm. Sci. Tech. 10 (2009) 443-452.

283 M. Li, 0. Rouaud, D. Poncelet. Microencapsulation by solvent evaporation: State of the art for process engineering approaches. Int. J. Pharm. 363 (2008) 26-39

284 A. A. Zaghloul. β-estradiol biodegradable microspheres: Effect of formulation parameters on encapsulation efficiency and in vitro release. Pharmazie 61 (2006) 775-779.

285 D. Chiappetta, M. J. Legaspi, V. Niselman, R. Pasquali, E. Gergic, A. C. Rodriguez Llimós, C. Bregni. Biodegradable microspheres of poly(D,L-lactide) containing progesterone. Ars Pharmaceutica 46 (2005) 383-398.

286 R. Jones, L. Mulamula, S. Swaminathan, Y. Tesema, D. Raghavan. Controlled release of β-estradiol from biodegradable polymeric microspheres. Mater. Res. Soc. Symp. Proc. 2004 EXS (1), art. 14.8, 429-431.

287 T. Mogi, N. Ohtake, M. Yoshida, R. Chimura, Y. Kamaga, S. Ando, T. Tsukamoto, K. Makino. Sustained release of 17β-estradiol from poly (lactide-co-glycolide) microspheres in vitro and in vivo. Colloids and Surfaces B: Biointerfaces 17 (2000) 153-165.

288 Q. Yang, G. Owusu-Ababio. Biodegradable progesterone microsphere delivery system for osteoporosis therapy. Drug Development and Industrial Pharmacy 26 (2000) 61-70.

289 H. K. Kim, T. G. Park. Microencapsulation of human growth hormone within biodegradable polyester microspheres: Protein aggregation stability and incomplete release mechanism. Biotech. Bioeng. 65 (1999) 659-667.

1. Injectable controlled release microparticle characterized in that comprises a polyvinyl alcohol polymer and at least one hormone.

2. The microparticle of claim 1 characterized in that the polyvinyl alcohol polymer having an hydrolysis degree of over 85%.

3. The microparticle of claim 1 characterized in that the polyvinyl alcohol polymer presents a degree of hydrolysis over 90%.

4. The microparticle of claim 1 characterized in that the polyvinyl alcohol polymer presents a degree of hydrolysis over 95%.

5. The microparticle of claim 1 characterized in that the polyvinyl alcohol polymer having a viscosity according to din 53015 protocol between 5 and 110 mpa.

6. The microparticle of claim 1 characterized in that the polyvinyl alcohol polymer presents a viscosity evaluated at a value between 20 and 70 mPa·s according to DIN 53015.

7. The microparticle of claim 1 characterized in that the polyvinyl alcohol polymer presents a viscosity evaluated at a value between 30 and 50 mPa·s according to DIN 53015.

8. The microparticle of claim 1 characterized in that said hormone is selected from a group comprising progesterone and its variants, aestradiol and its variants, prostaglandins and its variants, all the variants of prostanoic acid, steriods with progestagen activity, such as MGA melengestrol acetate, CAP (6-chloro-6-dehydro-17α-acetoxy-pregn-4-ene-3.20-dione). MAP (6α-methyl-17α-acetoxy-pregn-4-ene-3.20-dione); blocks of progestagens such as norgestomet, valerate aestradiol, benzoate aestradiol, 17 α aestradiol, gonadotropins such as GnRH, LH, CG, PMSG, FSH; and mixtures of said hormones.

9. The microparticle of claim 1 characterized in that said hormone is progesterone.

10. The microparticle of claim 1 characterized in that comprises a hormone concentration between 5 and 70% by weight of the total weight.

11. The microparticle of claim 1 characterized in that comprises a hormone concentration between 50% and 70% by weight of the total weight.

12. The microparticle of claim 1 characterized in that comprises a hormone concentration of at least 5% by weight of the total weight.

13. The microparticle of claim 1 characterized in that comprises a diameter of said microparticle between 0.2 to 5 mm.

14. The microparticle of claim 1 characterized in that comprises a diameter of 1.5 to 2.5 mm and dispersion in the diameters ranges from 0.01 to 0.1 mm.

15. The microparticle of claim 1 characterized in that comprises a diameter ranging from 1 to 2 mm when the hormone concentration is between 5 and 40% by weight, and a sphericity ranging from 1 to 1.5.

16. The microparticle of claim 1 characterized in that comprises a diameter ranging from 2 to 2.5 mm when the hormone load is between 40 and 50% in weight, dispersion in the diameter ranges from 0.01 to 0.1 mm, sphericity ranging from 1 to 1.5.

17. A process for producing the microparticle of claim 1 characterized in that comprises the following steps:

18. preparing an aqueous solution A, of polyvinyl alcohol and the hormone to be encapsulated with the optional adding of additives;

19. preparing a sodium hydroxide aqueous solution b, with the optional adding of additives.

20. dispersing solution A within solution B;

21. stabilising the microparticles formed in step c-, leaving them in suspension for a time period ranging from 2 to 90 minutes and at a temperature ranging between 20 and 90° C.;

22. recovering the microparticles, separating them from solution B;

23. drying the microparticles in hot air at a temperature ranging between 25 and 120° C. under conditions of a fluidised bed to attain the removal of the excess solvation water;

24. conditioning the microparticles.

25. The process of claim 17, characterized in that step a- for preparing an aqueous solution A consists in mixing PVA ranging between 5 and 50% by weight, glycerol between 0.05 and 1% by weight, boric acid ranging between 0.05 and 5% by weight, and progesterone ranging between 5 and 70% by weight, and stirring softly in a thermostated bath at a temperature ranging from 10 to 90° C. for 5 to 240 minutes until total dissolution of the PVA, BH and GL.

26. The process of claim 17, characterized in that step b- consists in preparing an aqueous saline solution (solution B) using sodium hydroxide ranging between 0.05 and 1% by weight.

27. The process of claim 17, characterized in that step c- for dispersing solution A in solution B consists in dripping solution A by gravity feed into solution B at a volumetric ratio ranging from 5 to 50 parts of solution B for each part of solution A.

28. The process of claim 17, characterized in that step c- for dispersing solution A in solution B is performed by dripping through a drip head and because the droplets of solution A are released from the drip head by gravity feed.

29. The process of claim 17, characterized in that step c- for dispersing solution A in solution B is performed by means of a drip head, and because solution A droplets are released by vibratory action.

30. The process of claim 22, characterized in that said vibration is induced mechanically.

31. The process of claim 22, characterized in that said vibration is induced by sound.

32. The process of claim 22, characterized in that the vibration is induced by means of electric or piezoelectric pumps activated with alternating currents.

33. The process of claim 17, characterized in that step c- for dispersing solution A in solution B is performed by means of a drip head and because solution A droplets are released with electrostatic assistance.

34. The process of claim 17, characterized in that step c- for dispersing solution A in solution B is performed by dripping through a drip head and because solution A droplets are released with the assistance of blowing with a gaseous current.

35. The process of claim 27, characterized in that the gaseous current is air.

36. The process of claim 17, characterized in that said process comprises an additional step of stabilization of the microparticles subsequent to step c-.

37. The process of claim 17, characterized in that step d- is performed within aqueous solution B during a stabilisation period, with the adding of stabilisers or stabilising agents.

38. The process of claim 17, characterized in that optional step d- for microparticle stabilisation is performed within aqueous solution B for 2 to 90 minutes at a temperature ranging between 20 and 90° C.

39. The process of claim 17, characterized in that the step for microparticle recovery is performed by a method selected from the group consisting in flotation, sedimentation, centrifugation, or filtration.

40. The process of claim 17, characterized in that the step for microparticle recovery is followed by washing said microparticle with a stabilising solution.

41. The process of claim 17, characterized in that the step of the microparticle recovery is subsequent to the step of the microparticle stabilization, and it consists in at least one of the following steps:

42. filtration, sedimentation, centrifugation, or flotation;

43. modification of the stabilising solution;

44. reduction of the stabilising solution's volume.

45. The process of claim 17, characterized in that the step of the microparticle recovery is performed by filtration.

46. The process of claim 17, characterized in that the step of the microparticle conditioning consists in at least 3 optional steps:

47. removing solvation water remaining in the microparticles by hot air drying at a temperature ranging from 25 to 120° C. under conditions of a fluidised bed,

48. dispersing the microparticles in 2-pyrrolydone,

49. spraying with an aqueous glycerol solution ranging between 10 and 60% in weight on the surface of the microparticles and subsequently exposing the microparticle to a temperature ranging from 100 to 120° C. and a pressure ranging from 20 to 100 bar.

50. The microparticle of claim 1 characterized in that it is administered to a female mammal to induce oestrus.

51. The microparticle of claim 1 characterized in that it is administered in a single application to a female mammal to induce oestrus.
